SEQUENCE LISTING (6 sequences)

SEQ ID NO: 1 - RNA, 22 nt, Artificial sequence
  Synthetic polynucleotide
  misc_feature: miR-433
uacggugagccugucauuauuc

SEQ ID NO: 2 - RNA, 22 nt, Artificial sequence
  Synthetic polynucleotide
  misc_feature: miR-200c
cgucuuacccagcaguguuugg

SEQ ID NO: 3 - DNA, 27 bp, Artificial sequence
  Synthetic polynucleotide
  misc_feature (1)..(1): Propylthiol-modified
aaaaaaaaaatccttggtgcccgagtg

SEQ ID NO: 4 - RNA, 23 nt, Artificial sequence
  Synthetic polynucleotide
  misc_feature: miR-106a
aaaagugcuuacagugcagguag

SEQ ID NO: 5 - RNA, 22 nt, Artificial sequence
  Synthetic polynucleotide
  misc_feature: miR-135a*
uauagggauuggagccguggcg

SEQ ID NO: 6 - RNA, 22 nt, Artificial sequence
  Synthetic polynucleotide
  misc_feature: miR-605
agaaggcacuaugagauuuaga

(19) United States
(12) Patent Application Publication
(10) Pub. No.: US 2018/0238889 A1
(43) Pub. Date: Aug. 23, 2018

(54) THE SCANO-miR PLATFORM IDENTIFIES A DISTINCT CIRCULATING MICRORNA SIGNATURE FOR THE DIAGNOSIS OF DISEASE

(71) Applicant: NORTHWESTERN UNIVERSITY, Evanston, IL (US)

(72) Inventors: Chad A. Mirkin, Wilmette, IL (US); Joshua J. Meeks, Western Springs, IL (US); C. Shad Thaxton, Chicago, IL (US); Ali Alhasan, Eastern Province (SA)

(21) Appl. No.: 15/752,527

(22) PCT Filed: Aug. 15, 2016
(86) PCT No.: PCT/US2016/047100

§ 371 Date: Feb. 13, 2018

Related U.S. Application Data
(60) Provisional application No. 62/205,184, filed on Aug. 14, 2015.

(51) Int. Cl. G01N 33/574; C12N 15/113; C07H 23/00

(52) U.S. Cl. CPC G01N 33/57434; C12N 15/113; G01N 2800/56; G01N 2800/50; C07H 23/00

(57) ABSTRACT

The disclosure relates to the identification of a novel molecular signature based on the differential expression of circulating microRNAs (miRNA) in serum samples specific to patients with clinically significant diseases or disorders, such as cancer.

### CROSS-REFERENCE TO RELATED APPLICATION

This application claims the benefit of U.S. Provisional Application Ser. No. 62/205,184, filed Aug. 14, 2015, the disclosure of which is incorporated herein by reference in its entirety.

### STATEMENT OF GOVERNMENT INTEREST

This invention was made with government support under U54 CA151880 awarded by the National Institutes of Health. The government has certain rights in the invention.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename; 2015-130_Seqlisting.txt; Size: 1,831 bytes; created: Aug. 15, 2016.

### FIELD OF THE INVENTION

The disclosure relates to the identification of a novel molecular signature based on the differential expression of circulating microRNAs (miRNA) in serum samples specific to patients with clinically significant diseases or disorders, such as cancer.

### BACKGROUND

Prostate cancer (PCa) is the most common noncutaneous malignancy among men in the United States and the second most common cause of cancer mortality.¹ Despite its prevalence, there are no specific accurate diagnostic and prognostic biomarkers. Indeed, although serum prostate specific antigen (PSA) concentration is used as a routine screening tool for prostate cancer, up to 11% of men with a PSA <2.0 ng/ml may still have prostate cancer and based on the serum level alone it is not possible to distinguish between high and low risk prostate cancers.² Due to the lack of specificity with PSA-based screening and harm associated with overtreatment and overdiagnosis, the United States Preventative Services Task Force has recommended that physicians do not routinely perform PSA-based prostate cancer screening.³⁻⁵ The major criticism associated with PSA based screening is “overtreatment.” This may be reduced by improved risk stratification; men with low (LR) and very-low risk (VLR) PCa can be monitored on active surveillance while those with intermediate and high risk (HR) PCa benefit from treatment [Schroder F H, et al. (2009) N. Engl. J. Med. 360(13):1320-1328; Epstein J I (2010) J. Urol. 183(2):433-440; Hugosson J, et al. (2010) Lancet Oncol. II(8):725-732; Schroder F H, et al. (2012) N. Engl. J. Med. 366(II):981-990; Conti S L, et al. (2009) J. Urol. 181(4):1628-1633]. Treatment can be avoided in almost 70% of men in active surveillance at 15 years of follow-up [Klotz L, et al. (2015) J. Clin. Oncol. 33(3):272-277]. Yet, many urologists and patients are reluctant to monitor their cancer on active surveillance due to concerns for delaying treatment or potentially missing treatment of aggressive cancer during a window of cure. Evidence for inadequacy of staging and risk stratification is demonstrated by the increase in Gleason Grade from Gleason 6 to 7 or higher in 40% of patients treated with radical prostatectomy (RP) [Bostwick D G, Myers R P, & Oesterling J E (1994) Semin. Surg. Oncol. 10(I):60-72; Isariyawongse B K, et al. (2008) Urology 72(4):882-886]. Thus, significant discrepancies between prostate needle biopsy and RP specimens may be attributed to diagnostic pitfalls as only 2% of the prostate is sampled with a biopsy [Chun F K, et al. (2010) Eur. Urol. 58(6):851-864]. Improved staging, which can result in reduction in overtreatment, patient anxiety, and biopsy-related complications, can be achieved by identifying unique molecular signatures capable of discriminating aggressive forms of PCa [Barbieri C E, et al. (2013) Eur. Urol. 64(4):567-576].

In an effort to separate diagnosis from treatment, active surveillance for men with low and very low risk prostate cancer, which combines PSA screening with rigorous scheduled prostate biopsies, has been implemented to decrease rates of over treatment.⁶⁻⁹ However, active surveillance is a potential option only in a very select group of men with low grade and low volume PCas.¹⁰ From studies of men that meet strict pathologic criteria to begin active surveillance, nearly 70% can avoid treatment over five years.¹¹ Yet, many urologists and patients are reluctant to “watch” their cancer on active surveillance due to concerns for delaying treatment or potentially missing treatment during a window of cure. Moreover, aggressive PCas are often undergraded at the time of diagnosis and may occur in more than 40% of prostate biopsies due to the limited accuracy of the prostate biopsy to detect the ultimate cancer grade and aggressiveness.^{12,13} Thus, significant discrepancies between prostatic needle biopsy and radical prostatectomy (RP) specimens may be attributed mainly to diagnostic pitfalls¹⁴ Resolving such screening paradigms can be achieved by identifying novel molecular signatures capable of discriminating aggressive forms of PCa, which could lead to avoiding unnecessary biopsies, patient anxiety, or biopsy-related complications.

Malignant transformation from a healthy cell to a cancerous cell is believed to occur through a step-wise accumulation of genetic and epigenetic events. Detection of molecular signatures that are indicative of such genetic changes would provide a means for early diagnosis of PCa. MicroRNAs (miRNA, miR) are critical gene regulatory elements that are present in stable forms in serum samples, and have emerged as potential non-invasive biomarkers for cancer diagnosis.¹⁵⁻¹⁹ Accumulative evidence shows that exosomes function as delivery vehicles to circulate miRNAs and transport them from primary cancer sites to distal ones while also shielding miRNAs from serum nucleases.²⁰ Unique changes in the expression levels of specific exosomal miRNAs are believed to be indicative of cancer types or physiological states, and are being explored as tissue-specific and stable biomarkers.²¹⁻²⁵ Therefore, serum exosomal miRNAs can be used as non-invasive biomarkers to identify molecular signatures specific to insignificant PCa, aggressive PCa, or highly aggressive PCa.

With the potential that miRNAs hold as biomarkers, there are numerous challenges associated with profiling circulating miRNAs such as the short length of miRNAs (19-25 nucleotides), the existence of sequence similarity between miRNA family members, degradative enzymes, and the presence of these biomarkers at extremely low concentrations in serum samples.²⁶ Current methods for circulating miRNA profiling include conventional miRNA microarrays, deep sequencing, and quantitative real time PCR (qRT-PCR).

### SUMMARY

The Scano-miR system^{26,27} is capable of quantitatively profiling circulating miRNAs with high specificity and high sensitivity in a high-throughput fashion. Indeed, this assay, which does not rely on target enzymatic amplification and is therefore amenable to massive multiplexing, can detect such non-invasive biomarkers down to femtomolar concentration with the capability to distinguish perfect miRNA sequences from those with single nucleotide mismatches (i.e. SNPs).²⁷ The Scano-miR platform relies on the unique properties of spherical nucleic acids (SNAs) such as their high binding constant to target biomolecules and the amplifiable light scattering properties of gold nanoparticles to achieve high assay sensitivity.²⁸⁻³¹ In addition, these nanoconjugates exhibit elevated melting temperatures with sharp melting transitions relative to oligonucleotide duplexes formed from traditional DNA probes of the same sequence, which can be translated into significantly higher assay specificity.^{27,29} These attributes overcome many of the limitations of enzymatic amplification processes such as PCR, most notably the inability to screen a sample for 1000 s of miRNA targets without the need to individually amplify each of the targets.

Prostate cancer (PCa) is the most common noncutaneous malignancy among men in the United States and the second most common cause of cancer mortality. Despite its prevalence, there are no specific accurate diagnostic and prognostic biomarkers. Indeed, although serum prostate specific antigen (PSA) concentration is used as a routine screening tool for prostate cancer, up to 11% of men with a PSA <2.0 ng/ml may still have prostate cancer and based on the serum level alone it is not possible to distinguish between high and low risk prostate cancers. Due to the lack of specificity with PSA-based screening and harm associated with overtreatment and overdiagnosis, the United States Preventative Services Task Force has recommended that physicians do not routinely perform PSA-based prostate cancer screening. In an effort to separate diagnosis from treatment, active surveillance for men with low and very low risk prostate cancer, which combines PSA screening with rigorous scheduled prostate biopsies, has been implemented to decrease rates of over treatment. However, active surveillance is a potential option only in a very select group of men with low grade and low volume PCas. From studies of men that meet strict pathologic criteria to begin active surveillance, nearly 70% can avoid treatment over five years. Yet, many urologists and patients are reluctant to “watch” their cancer on active surveillance due to concerns for delaying treatment or potentially missing treatment during a window of cure. Moreover, aggressive PCas are often undergraded at the time of diagnosis and may occur in more than 40% of prostate biopsies due to the limited accuracy of the prostate biopsy to detect the ultimate cancer grade and aggressiveness. Thus, significant discrepancies between prostatic needle biopsy and radical prostatectomy (RP) specimens may be attributed mainly to diagnostic pitfalls. Resolving such screening paradigms can be achieved by identifying novel molecular signatures capable of discriminating aggressive forms of PCa, which could lead to avoiding unnecessary biopsies, patient anxiety, or biopsy-related complications.

The disclosure provides the ability to identify a novel molecular signature based on the differential expressions of circulating microRNAs (miRNA) in serum samples specific to patients with clinically significant cancer, such as prostate cancer (PCa). The Scano-miR platform was used to study the circulating miRNA profiles from patients with aggressive forms of PCa and to compare them with those from healthy individuals and ones with indolent forms of the disease. The data provided herein show potential biomarkers of five miRNAs that were confirmed using qRT-PCR on a validation set of 28 serum samples from blinded patients. Therefore, in some embodiments this molecular signature is used in clinical settings to diagnose patients with highly aggressive PCa. In further embodiments, the molecular signature is used in clinical settings to diagnose patients with very high risk PCa.

Thus, in some aspects the disclosure provides a method of determining a profile of microRNA (miRNA) comprising: isolating the miRNA from a sample; ligating the miRNA to a universal linker; hybridizing the miRNA to a nucleic acid that is on a surface, wherein the nucleic acid is complementary to the miRNA; contacting the miRNA with a spherical nucleic acid (SNA), wherein the SNA comprises a polynucleotide that is sufficiently complementary to the universal linker to hybridize under appropriate conditions; and detecting the SNA to determine the miRNA profile.

In further aspects, the disclosure provides a method of detecting aggressive prostate cancer in an individual, the method comprising: isolating miRNA from a sample from the individual; ligating the miRNA to a universal linker; hybridizing the miRNA to a nucleic acid that is on a surface, wherein the nucleic acid is complementary to miR-433 (SEQ ID NO: 1) and/or miR-200c (SEQ ID NO: 2); contacting the miRNA with a spherical nucleic acid (SNA), wherein the SNA comprises a polynucleotide that is sufficiently complementary to the universal linker to hybridize under appropriate conditions; wherein detection of the SNA is indicative of aggressive prostate cancer in the individual.

In some embodiments, the SNA comprises a metal. In related embodiments, the SNA comprises gold. In further embodiments, the SNA is hollow. In still further embodiments, the SNA comprises a liposome.

In some embodiments, the surface is an array. In further embodiments, the array comprises a plurality of different nucleic acids.

In some embodiments, the sample is a body fluid, serum, or tissue obtained from an individual suffering from a disease. In further embodiments, the sample is a body fluid, serum, or tissue obtained from an individual not known to be suffering from a disease. In related embodiments, the body fluid is saliva, urine, plasma, cerebrospinal fluid (CSF), bile, breast milk, feces, gastric juice, mucus, peritoneal fluid, sputum, sweat, tears, or a vaginal secretion.

In any of the embodiments of the disclosure, the sample is a liquid/fluid biopsy. Liquid biopsy is advantageous over tissue biopsy, because it is less invasive to obtain a liquid sample from the patient or subject, and liquid biopsy overcomes some of the issues of tumor heterogeneity associated with tissue biopsy; information acquired from a single biopsy provides a spatially and temporally limited snap-shot of a tumor that does not necessarily reflect its heterogeneity. A liquid biopsy provides the genetic landscape of all cancerous lesions (primary and metastases) as well as offering the opportunity to systemically track genomic evolution [Crowley et al., Nat. Rev. Clin. Oncol. 10(8): 472 (2013)]. Examples of liquid biopsy samples include blood samples and/or nipple aspirates. The sample is, in various embodiments, one or more blood samples taken from a patient undergoing therapy.

In some embodiments, the profile is compared to an earlier profile determined from the individual. In further embodiments, the profile is compared to a profile determined from an additional individual known to be suffering from a disease.

In some embodiments, the disease is cancer. In related embodiments, the cancer is a hematological tumor or a solid tumor. In still further embodiments, the cancer is bladder cancer, brain cancer, cervical cancer, colon/rectal cancer, leukemia, lymphoma, liver cancer, ovarian cancer, pancreatic cancer, sarcoma, prostate cancer, or breast cancer.

In some embodiments, the disease is an inflammatory disorder or an auto-immune disease. In further embodiments, the inflammatory disorder is infectious or sterile.

In some embodiments, the miRNA is exosomal miRNA. In further embodiments, the nucleic acid is complementary to miR-433 (5′-uacggugagccugucauuauuc-3′ (SEQ ID NO: 1)) and/or miR-200c (5′-cgucuuacccagcaguguuugg-3′ (SEQ ID NO: 2)) and the profile indicates aggressive prostate cancer. In some embodiments, the profile indicates very high risk prostate cancer.

In further aspects, the disclosure provides a method of detecting aggressive prostate cancer in an individual, the method comprising: isolating miRNA from a sample from the individual; ligating the miRNA to a universal linker; hybridizing the miRNA to a nucleic acid that is on a surface, wherein the nucleic acid is complementary to miR-433 (SEQ ID NO: 1), miR-106a (5′-aaaagugcuuacagugcagguag-3′ (SEQ ID NO: 4)), miR-135a* (5′-uauagggauuggagccguggcg-3′ (SEQ ID NO: 5)), miR-605 (5′-agaaggcacuaugagauuuaga-3′ (SEQ ID NO: 6)), and/or miR-200c (SEQ ID NO: 2); contacting the miRNA with a spherical nucleic acid (SNA), wherein the SNA comprises a polynucleotide that is sufficiently complementary to the universal linker to hybridize under appropriate conditions; wherein detection of the SNA is indicative of aggressive prostate cancer in the individual. In some embodiments, the aggressive prostate cancer is very high risk prostate cancer. In some embodiments, the SNA comprises a metal. In related embodiments, the SNA comprises gold. In some embodiments, the SNA is hollow. In further embodiments, the SNA comprises a liposome.

In some embodiments, the surface is an array. In related embodiments, the array comprises a plurality of different nucleic acids.

In some embodiments, the sample is a body fluid, serum, or tissue obtained from an individual suffering from a disease. In some embodiments, the sample is a liquid biopsy from an individual suffering from a disease.

In further embodiments, the sample is a body fluid, serum, or tissue obtained from an individual not known to be suffering from a disease. In some embodiments, the sample is a liquid biopsy from an individual not known to be suffering from a disease.

In some embodiments, the profile is compared to an earlier profile determined from the individual. In further embodiments, the profile is compared to a profile determined from an additional individual known to be suffering from aggressive prostate cancer. In various embodiments, the miRNA is exosomal miRNA. In any of the aspects or embodiments of the disclosure, the aggressive prostate cancer is very high risk prostate cancer.

Herein, the Scano-miR platform is used to study the exosomal miRNA profiles of serum samples from patients with aggressive forms of PCa and compare them with the serum sample miRNA profiles from healthy individuals and ones with indolent forms of the disease. The data show significant changes in the expression levels of two up-regulated miRNAs and two down-regulated miRNAs in addition to one exclusively expressed miRNA in highly aggressive forms of PCa. Moreover, the identified molecular signature that consists of differentially co-expressed miRNAs exhibits a high correlation to the clinical pathology of patients identified with varying degrees of PCa. In addition, individual miRNAs were found that distinguished between patients with indolent versus highly aggressive PCa (miR-433) and between patients with highly aggressive versus normal or indolent PCa (miR-200c). Therefore, in some embodiments the disclosure provides a novel molecular signature for the diagnosis and prognosis of aggressive PCa. In further embodiments the disclosure provides a novel molecular signature for the diagnosis and prognosis of very high risk PCa.

### BRIEF DESCRIPTION OF THE FIGURES

FIG. 1 depicts a heat map of all co-expressed miRNAs. Hierarchical clustering was performed on 45 co-expressed microRNA using the Pearson correlation metric. Included microRNA are expressed to some extent in aggressive (n=8) and control samples (n=8). Samples from patients with aggressive PCa generally cluster together and have globally upregulated serum microRNA expression.

FIG. 2 shows differentially expressed miRNAs. Boxplots represent the background subtracted, normalized distributions of 6 differentially expressed miRNAs. The red bar represents the median, while the blue bar represents the interquartile range of distribution. (Permutation t-test; miR-106a, p=0.0018; miR-371-3p, p=0.0089; miR-433, p=0.0115; miR=605, p=0.0301; miR-135a*, p=0.0319; miR-495, p=0.0411).

FIG. 3 shows the Molecular Signature Score of 6 miRNAs. The molecular signature score was calculated for the 6 differentially expressed miRNAs using the procedure described in Zeng et al (2012). Distinct ranges of the combined intensity score shows that there is little overlap between aggressive and control group expression when using an aggregate score. (p=0.0036).

FIG. 4 depicts a heat map of clustering and clinical association for 6 differentially expressed miRNAs. Unsupervised hierarchical clustering performed on expression profiles for 16 serum samples reveals that generally samples of similar histology are clustered together. Interestingly, a subgroup of 4 samples identified to be indicative of highly aggressive prostate cancer cluster together using this molecular signature. Gleason scores are on the range of 9 (black) to 6 (light gray) to N/A (white). Tumor staging is on the range of T3 (black) to T1 (light gray) to N/A (white). Risk status scale is VHR—very high risk, HR—high risk, LR—low risk, or healthy. Patients were categorized based on the 2015 NCCN Guidelines for Prostate Cancer (Version 1.2015).

FIG. 5 shows successful validation of five miRNAs (miR-200c, miR-605, miR-135a*, miR-433, and miR-106a) using qRT-PCR from blinded patients showing distinct patterns that correlate with healthy specimens, aggressive PCa, or indolent PCa.

FIG. 6 shows qRT-PCR analysis of blinded patients successfully validated five miRNAs (miR-200c, miR-605, miR-135a*, miR-433, and miR-106a), whereas two miRNAs (miR-495 and miR-371-3p) showed no detectable signals across all samples. Molecular signature score of co-expressed miRNAs (miR-605, miR-135a*, miR-433, and miR-106a) in indolent and aggressive PCas significantly distinguishes clinically significant cancer from indolent (p<0.0001, FIG. 6E).

FIG. 7 shows relative expression levels of significantly deregulated miR-106a, -135a*, -433, and -605 (fold change >1.5).

FIG. 8 shows qRT-PCR validation of the Blinded Samples (8a-8d) Blinded qRT-PCR analysis of patient serum samples successfully validated four co-expressed miRNAs (miR-605, miR-135a*, miR-433, and miR-106a) (fold change >1.5). FIG. 8e) Blinded qRT-PCR analysis of a validated, exclusively expressed miRNA; miR-200c.

FIG. 9 depicts the Specificity and Sensitivity Analysis. Receiver operating characteristic (ROC) curves were generated to compare the ROC of the Scano-miR miRNAs (a-e) to the Gleason sum from 1st prostatic needle biopsy (FB) (f). The miRNAs identified by the Scano-miR bioassay are at least 89.5% accurate in differentiating between VHR PCa versus control group.

FIG. 10 depicts KEGG Pathway Analysis of the Validated miRNAs. Target genes and biological pathways for upregulated miRNAs (red ovals (miR-433, miR-200c, and miR-106a)) and downregulated miRNAs (green oval (miR-135A* and miR-605)) were identified using microT-CDS and TarBase to classify the Gene Ontology (GO) category and KEGG pathway enrichment with a corrected p-value threshold of <0.05. The yellow squares (TGFA, PDGFA, IGF1R, PIK3CA, GRB2, SOS1, PTEN, MDM2, CDKN1A, CASP9, RB1, LEF1, CREBS, TP53, NFKB1, E2F1, CCND1, BCL2 and MAPK1) represent target genes potentially altered by the expression of the validated miRNAs, and blue squares (Ras, Raf, PIP3, MEK, PKB/Akt, B-Catenin, GSK3B and IKK) represent genes that are not directly targeted by the validated miRNAs.

### DESCRIPTION

Detection of molecular signatures that are indicative of molecular processes related to aggressive forms of PCa allows biological insight into differentiating aggressive from indolent PCa. MicroRNAs (miRNA, miR) are critical gene regulatory elements that are present in stable forms in serum and have emerged as potential non-invasive biomarkers for cancer diagnosis [Lee R C, Feinbaum R L, & AmbrosV (1993) Cell 75(5):843-854; Lim L P, et al. (2005) Nature 433(7027): 769-773; Lewis B P, Burge C B, & Bartel D P (2005) Cell 120(I):15-20; Mitchell P S, et al. (2008) Proc. Natl. Acad. Sci. USA 105(30):10513-10518; Selth L A, et al. (2012) Int. J. Cancer 131(3):652-661]. Exosomes are thought to function as delivery vehicles of circulating miRNAs and transport them from primary cancer sites to metastatic sites while also shielding miRNAs from serum nucleases [Valadi H, et al. (2007) Nat. Cell Biol. 9(6):654-659; Alhasan A H, Patel P C, Choi C H J, & Mirkin C A (2014) Small 10(1)186-192]. Therefore, serum exosomal miRNAs serve as non-invasive biomarkers to identify molecular signatures specific to patients with a higher risk of developing aggressive forms of PCa relative to those with indolent PCa. Others have identified miRNA signatures and linked them to PCa progression. Circulating miR-141, miR-200c, and miR-375 have been proposed as potential blood markers for the diagnosis of PCa [Mitchell P S, et al. (2008) Proc. Natl. Acad. Sci. USA 105(30):10513-10518; Brase J C, et al. (2011) Int. J. Cancer. 128(3):608-616; WatahikiA, et al. (2013) Int. J. Mol. Sci. 14(4): 7757-7770]. However, the heterogeneity of PCas do not allow intermediate grades of PCa to be distinguished from aggressive forms using these previously identified miRNA signatures. In the present disclosure, however, determination of the miRNA expression pattern of very high risk (VHR) PCa is provided, and the expression pattern was validated in men with differing PCa aggressiveness.

The Scano-miR platform was used to study the exosomal miRNA profiles of serum samples from patients with aggressive forms of prostate cancer (PCa) and compare them with the serum sample miRNA profiles from healthy individuals and ones with indolent forms of the disease. The data show significant changes in the expression levels of two up-regulated miRNAs and two down-regulated miRNAs in addition to one exclusively expressed miRNA in highly aggressive forms of PCa. Moreover, the identified molecular signature that consists of differentially co-expressed miRNAs exhibits a high correlation to the clinical pathology of patients identified with varying degrees of PCa. In addition, individual miRNAs were found that distinguished between patients with indolent versus highly aggressive PCa (miR-433) and between patients with highly aggressive versus normal or indolent PCa (miR-200c). Therefore, in some aspects, the disclosure provides a molecular signature for the diagnosis and prognosis of aggressive PCa. In further embodiments the disclosure provides a novel molecular signature for the diagnosis and prognosis of very high risk PCa.

Serum microRNAs (miRNAs) have emerged as potential noninvasive biomarkers to diagnose prostate cancer (PCa), the most common noncutaneous malignancy among western men. However, intermediate grades of PCa cannot be distinguished from aggressive forms using current miRNA signatures due to the heterogeneity of PCas. Recently, a high-throughput, spherical nucleic acid (SNA)-based miRNA expression profiling platform, called the Scano-miR bioassay, was developed to measure the expression levels of miRNAs with both high sensitivity and specificity. By studying serum miRNAs of PCa using the Scano-miR bioassay a unique molecular signature specific for very high-risk aggressive PCa has been identified and is disclosed herein. This molecular signature will assist in differentiating patients that may benefit from therapy from those that can be closely monitored on active surveillance.

It is noted here that, as used in this specification and the appended claims, the singular forms “a,” “an,” and “the” include plural reference unless the context clearly dictates otherwise.

As used herein, the term “polynucleotide,” is used interchangeably with the term oligonucleotide and the terms have meanings accepted in the art.

It is further noted that the terms “attached”, “conjugated” and “functionalized” are also used interchangeably herein and refer to the association of a polynucleotide with a nanoparticle.

“Hybridization” means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art.

As used herein “aggressive prostate cancer” refers to patients with a high Gleason score prostate cancer GS 7). Aggressive prostate cancer includes two risk groups, high risk and very high risk.

As used herein, “Very high risk” prostate cancer is defined according to the 2015 National Comprehensive Cancer Network (NCCN) Guidelines for Prostate Cancer. According to the 2015 NCCN guidelines, individuals with “very high risk” prostate cancer refer to those with a T3b or T4 tumor, primary Gleason grade 5, or more than 4 biopsy cores with Gleason scores between 8 and 10.

Scanometric Assay.

The scanometric assay is a nucleic acid detection method originally based upon the use of spherical nucleic acid-gold nanoparticle conjugates (SNA-Au NPs) [Taton et al., Science 289:1757 (2000); Mirkin et al., Nature 382:607 (1996); Rosi et al., Science 312:1027. (2006); Prigodich et al., J. Am. Chem. Soc. 133:2120. (2011); Hao et al., Small. 7(22):3158 (2011); Cutler et al., J. Am. Chem. Soc. 134:1376 (2012)]. The assay utilizes a low density microarray on a glass slide to capture DNA target and then sandwiches it with the SNA-Au NP probes. The signal is then amplified by catalytic reduction of Ag+ in the presence of hydroquinone [Taton et al., Science. 289:1757 (2000)] or gold enhancement with tetrachloroaurate and hydroxylamine [Kim et al., Anal. Chem. 81:9183. (2009); Ma et al., Angew. Chem. Int. Ed. 41:2176 (2002)]. After the reduction step, the slide is used as a wave guide, and scattered light is measured from the metal spots to determine target identity and concentration. The LOD of the method is 100 aM for large DNA targets and does not require PCR or related target amplification techniques [Cao et al., Science. 297:1536 (2002)]. Because the SNA-Au NP probes exhibit cooperative melting transitions over more narrow temperature ranges than duplexes formed from molecular fluorophore probes of the same sequence, stringency conditions can be employed to provide significantly higher target discrimination capability [Taton et al., Science. 289:1757 (2000)].

Herein it is shown that this assay is ideal for detecting short, relatively low abundance miRNAs (i.e., Scano-miR assay), without the need for enzymatic amplification steps with high selectivity and sensitivity. Thus, the methods herein are directed to profiling the expression of miRNA species from a sample, e.g., human serum, cell culture, and human tissue samples. The Scano-miR assay is highly specific, sensitive, and reproducible for profiling miRNAs. Importantly, this scanometric method can be used not only with high density arrays but it can also identify miRNA markers with higher sensitivity and selectivity than fluorophore based high-density array techniques.

Spherical Nucleic Acids.

Spherical nucleic acids (SNAs) comprise densely functionalized and highly oriented polynucleotides on the surface of a nanoparticle which can either be inorganic (such as gold, silver, or platinum) or hollow (such as liposomal or silica-based). The spherical architecture of the polynucleotide shell confers unique advantages over traditional nucleic acid delivery methods, including entry into nearly all cells independent of transfection agents and resistance to nuclease degradation. Furthermore, SNAs can penetrate biological barriers, including the blood-brain and blood-tumor barriers as well as the epidermis.

Nanoparticles are therefore provided which are functionalized to have a polynucleotide attached thereto. In general, nanoparticles contemplated include any compound or substance with a high loading capacity for a polynucleotide as described herein, including for example and without limitation, a metal, a semiconductor, a liposomal particle, insulator particle compositions, and a dendrimer (organic versus inorganic).

Thus, nanoparticles are contemplated which comprise a variety of inorganic materials including, but not limited to, metals, semi-conductor materials or ceramics as described in US patent application No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use in producing nanoparticles.

Liposomal particles, for example as disclosed in PCT/US2014/068429 (incorporated by reference herein in its entirety) are also contemplated by the disclosure. Hollow particles, for example as described in U.S. Patent Publication Number 2012/0282186 (incorporated by reference herein in its entirety) are also contemplated herein.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO2, Sn, SnO2, Si, SiO2, Fe, Fe+4, Ag, Cu, Ni, Al, steel, cobalt-chrome alloys, Cd, titanium alloys, AgI, AgBr, HgI2, PbS, PbSe, ZnTe, CdTe, In2S3, In2Se3, Cd3P2, Cd3As2, InAs, and GaAs. Methods of making ZnS, ZnO, TiO2, AgI, AgBr, HgI2, PbS, PbSe, ZnTe, CdTe, In2S3, In2Se3, Cd3P2, Cd3As2, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshaysky, et al., J. Am. Chem. Soc., 112, 9438 (1990); Ushida et al., J. Phys. Chem., 95, 5382 (1992).

In practice, methods of increasing cellular uptake and inhibiting gene expression are provided using any suitable particle having oligonucleotides attached thereto that do not interfere with complex formation, i.e., hybridization to a target polynucleotide. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles particles, aggregate particles, isotropic (such as spherical particles) and anisotropic particles (such as non-spherical rods, tetrahedral, prisms) and core-shell particles such as the ones described in U.S. patent application Ser. No. 10/034,451, filed Dec. 28, 2002 and International application no. PCT/US01/50825, filed Dec. 28, 2002, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preaparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers)

Suitable nanoparticles are also commercially available from, for example, Ted Pella, Inc. (gold), Amersham Corporation (gold) and Nanoprobes, Inc. (gold).

Also as described in US patent application No 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in US patent application No 20030147966, nanoparticles contemplated are produced using HAuCl4 and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or the amount of surface area that can be functionalized as described herein.

Polynucleotides.

The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. No. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or a modified form thereof generally comprise a polynucleotide from about 5 nucleotides to about 100 nucleotides in length. More specifically, nanoparticles are functionalized with a polynucleotide that is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 125, about 150, about 175, about 200, about 250, about 300, about 350, about 400, about 450, about 500 or more nucleotides in length are contemplated.

In some embodiments, the polynucleotide attached to a nanoparticle is DNA. When DNA is attached to the nanoparticle, the DNA is in some embodiments comprised of a sequence that is sufficiently complementary to a target region of a polynucleotide such that hybridization of the DNA polynucleotide attached to a nanoparticle and the target polynucleotide (e.g., a miRNA target) takes place, thereby associating the target polynucleotide to the nanoparticle. The DNA in various aspects is single stranded or double-stranded, as long as the double-stranded molecule also includes a single strand region that hybridizes to a single strand region of the target polynucleotide. In some aspects, hybridization of the polynucleotide functionalized on the nanoparticle can form a triplex structure with a double-stranded target polynucleotide. In another aspect, a triplex structure can be formed by hybridization of a double-stranded oligonucleotide functionalized on a nanoparticle to a single-stranded target polynucleotide.

In some embodiments, the disclosure contemplates that a polynucleotide attached to a nanoparticle is RNA. The RNA can be either single-stranded or double-stranded, so long as it is able to hybridize to a target polynucleotide.

In some aspects, multiple polynucleotides are functionalized to a nanoparticle. In various aspects, the multiple polynucleotides each have the same sequence, while in other aspects one or more polynucleotides have a different sequence. In further aspects, multiple polynucleotides are arranged in tandem and are separated by a spacer. Spacers are described in more detail herein below.

Polynucleotide Attachment to a Nanoparticle.

Polynucleotides contemplated for use in the methods include those bound to the nanoparticle through any means. Regardless of the means by which the polynucleotide is attached to the nanoparticle, attachment in various aspects is effected through a 5′ linkage, a 3′ linkage, some type of internal linkage, or any combination of these attachments.

Methods of attachment are known to those of ordinary skill in the art and are described in US Publication No. 2009/0209629, which is incorporated by reference herein in its entirety. Methods of attaching RNA to a nanoparticle are generally described in PCT/US2009/65822, which is incorporated by reference herein in its entirety. Methods of associating polynucleotides with a liposomal particle are described in PCT/US2014/068429, which is incorporated by reference herein in its entirety.

Spacers.

In certain aspects, functionalized nanoparticles are contemplated which include those wherein an oligonucleotide and a domain are attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotides in tandem, whether the oligonucleotides have the same sequence or have different sequences. In aspects of the invention where a domain is attached directly to a nanoparticle, the domain is optionally functionalized to the nanoparticle through a spacer. In another aspect, the domain is on the end of the oligonucleotide that is opposite to the spacer end. In aspects wherein domains in tandem are functionalized to a nanoparticle, spacers are optionally between some or all of the domain units in the tandem structure. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, an ethylglycol, or combinations thereof.

In certain aspects, the polynucleotide has a spacer through which it is covalently bound to the nanoparticles. These polynucleotides are the same polynucleotides as described above. As a result of the binding of the spacer to the nanoparticles, the polynucleotide is spaced away from the surface of the nanoparticles and is more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the polynucleotides to become bound to the nanoparticles or to the target polynucleotide. In certain aspects, the bases of the polynucleotide spacer are all adenylic acids, all thymidylic acids, all cytidylic acids, all guanylic acids, all uridylic acids, or all some other modified base. Accordingly, in some aspects wherein the spacer consists of all guanylic acids, it is contemplated that the spacer can function as a domain as described herein.

Nanoparticle Surface Density.

A surface density adequate to make the nanoparticles stable and the conditions necessary to obtain it for a desired combination of nanoparticles and polynucleotides can be determined empirically. Generally, a surface density of at least about 2 pmoles/cm² will be adequate to provide stable nanoparticle-oligonucleotide compositions. In some aspects, the surface density is at least 15 pmoles/cm². Methods are also provided wherein the polynucleotide is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm2, at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Surface.

The surface can be any material to which species of miRNA may be attached, e.g., glass. As disclosed herein, in some aspects the surface is a microarray. The microarray can be either a low density or high density microarray. In some embodiments, the microarray is a commercially-available microarray that displays a complement of a miRNA of interest.

miRNA Target Polynucleotide.

As disclosed herein, in any of the aspects or embodiments of the disclosure, the target polynucleotide is miRNA. Non-limiting examples of target miRNAs are any of the miRNAs disclosed herein, including miR-200c, miR-605, miR-135a*, miR-433, and miR-106a.

Relationship of miRNA Profile with Disease Progression.

As exemplified herein, certain miRNA profiles are indicative of particular disease states. By way of example, in prostate cancer samples, miR-200c was shown herein to only be detected in serum samples from patients with highly aggressive prostate cancer (PCa) and miR-433 was found to be differentially expressed in highly aggressive versus indolent PCa serum samples.

Thus, identification of a patient's miRNA according to the methods disclosed herein provides a molecular signature for the diagnosis and prognosis of aggressive PCa.

Additional aspects and embodiments of the disclosure are described in the following enumerated paragraphs.

Paragraph 1.

A method of determining a profile of microRNA (miRNA) comprising: isolating the miRNA from a sample; ligating the miRNA to a universal linker; hybridizing the miRNA to a surface comprising a nucleic acid that is complementary to the miRNA; contacting the miRNA with a spherical nucleic acid (SNA), wherein the SNA comprises a polynucleotide that is sufficiently complementary to the universal linker to hybridize under appropriate conditions; and detecting the SNA to determine the miRNA profile.

Paragraph 2.

The method of paragraph 1 wherein the SNA comprises a metal.

Paragraph 3.

The method of paragraph 2 wherein the SNA comprises gold.

Paragraph 4.

The method of paragraph 1 wherein the SNA is hollow.

Paragraph 5.

The method of paragraph 1 wherein the SNA comprises a liposome.

Paragraph 6.

The method of any one of paragraphs 1-5 wherein the surface is an array.

Paragraph 7.

The method of paragraph 6 wherein the array comprises a plurality of different nucleic acids.

Paragraph 8.

The method of any one of paragraphs 1-7 wherein the sample is a body fluid (including but not limited to saliva, urine, plasma, cerebrospinal fluid (CSF), bile, breast milk, feces, gastric juice, mucus, peritoneal fluid, sputum, sweat, tears, and a vaginal secretion), serum, or tissue obtained from an individual suffering from a disease.

Paragraph 9.

The method of any one of paragraphs 1-7 wherein the sample is a body fluid (including but not limited to saliva, urine, plasma, cerebrospinal fluid (CSF), bile, breast milk, feces, gastric juice, mucus, peritoneal fluid, sputum, sweat, tears, and a vaginal secretion), serum, or tissue obtained from an individual not known to be suffering from a disease.

Paragraph 10.

The method of paragraph 8 wherein the profile is compared to an earlier profile determined from the individual.

Paragraph 11.

The method of paragraph 9 wherein the profile is compared to a profile determined from an additional individual known to be suffering from a disease.

Paragraph 12.

The method of any one of paragraphs 8, 10 or 11 wherein the disease is cancer.

Paragraph 13.

The method of paragraph 12 wherein the cancer is a hematological tumor or a solid tumor.

Paragraph 14.

The method of paragraph 12 or paragraph 13 wherein the cancer is bladder cancer, brain cancer, cervical cancer, colon/rectal cancer, leukemia, lymphoma, liver cancer, ovarian cancer, pancreatic cancer, sarcoma, prostate cancer, or breast cancer.

Paragraph 15.

The method of any one of paragraphs 8, 10, or 11 wherein the disease is an inflammatory disorder or an auto-immune disease.

Paragraph 16.

The method of paragraph 15 wherein the inflammatory disorder is infectious or sterile.

Paragraph 17.

The method of any one of paragraphs 1-16 wherein the miRNA is exosomal miRNA.

### EXAMPLES

The Scano-miR platform was used to study the circulating miRNA profiles from patients with aggressive forms of PCa and to compare them with those from healthy individuals and ones with indolent forms of the disease. The data show potential biomarkers of five miRNAs (miR-200c, miR-605, miR-135a*, miR-433, and miR-106a) that were confirmed using qRT-PCR on a validation set of 28 serum samples from blinded patients. Importantly, miR-200c was only detected in serum samples from patients with highly aggressive PCa, whereas miR-433 was differentially expressed in aggressive versus indolent PCa and undetected in healthy individuals. Therefore, this molecular signature is useful in clinical settings to diagnose patients with highly aggressive PCa with at least 94% accuracy.

Circulating microRNA Profiling Using the Scano-miR Bioassay.

Current methods for miRNA profiling include miRNA fluorophore-based microarray techniques, deep sequencing, quantitative real time PCR (qRT-PCR), and more recently, techniques based upon spherical nucleic acid (SNA) gold nanoparticle conjugates and the Scano-miR platform [Grasedieck S, et al. (2013) Blood 121(25):4977-4984; Mirkin C A, Letsinger R L, Mucic R C, & Storhoff J J (1996) Nature 382(6592):607-609; Taton T A, Mirkin C A, & Letsinger R L (2000) Science 289(5485):1757-1760; Alhasan A H, et al. (2012) Anal. Chem. 84(9):4153-4160]. The Scano-miR bioassay, which does not rely on target enzymatic amplification and is therefore amenable to massive multiplexing to screen a sample for thousands of relatively short miRNA targets (19-25 nucleotides), can detect miRNA biomarkers down to 1 femtomolar concentrations with the capability to distinguish perfect miRNA sequences from those with single nucleotide mismatches (i.e. SNPs) [Alhasan A H, et al. (2012) Anal. Chem. 84(9):4153-4160]. The Scano-miR platform was used herein to study the exosomal miRNA profiles of serum samples from patients with VHR PCa and compared with the miRNA profiles from healthy individuals and ones with LR PCa.

### Example 1

To identify a novel molecular signature capable of detecting aggressive PCa using the Scano-miR platform, a training set of 16 serum samples were obtained from healthy donors and patients with varying grades of PCa (Table 1 and Table 2). In a typical Scano-miR assay, exosomes were isolated from serum samples followed by miRNA extraction and ligation to a universal miRNA cloning linker.²⁷ The ligation mixtures from each serum sample were hybridized directly onto separate miRNA microarrays (miR-array) (NCode Human miRNA Microarray V3, Invitrogen). To profile the miRNA expression, a universal SNA probe was synthesized by chemisorbing DNA sequences complementary to the miRNA cloning linker onto gold nanoparticles. The SNAs were added to the miR-arrays in order to bind the ligated miRNA species. Finally, a gold enhancement solution consisting of HAuCl₄ and NH₂OH [Alhasan A H, et al. (2012) Anal. Chem. 84(9):4153-4160; Kim D, Daniel W L, & Mirkin C A (2009) Anal. Chem. 81(21):9183-9187] was added in order to enhance the scattered light signals from the SNA probes and to detect low abundance serum miRNAs. These signals were measured with a Tecan LS Reloaded Scanner and used to extract the miRNA profiles and to determine the miRNA expression levels from each serum sample.

TABLE 1
Clinical annotation for n = 16 patients screened for
PCa. The Gleason Score is the combined Gleason score obtained
through biopsy and histological examination. Clinical tumor
stage and cancer staging was based on pathological examination
of the primary tumor. VHR—very high risk, HR—high risk,
LR—low risk. Patients were categorized based on the 2015
NCCN Guidelines for Prostate Cancer (Version 1.2015)

| | Clinical | Clinical | |
|---|---|---|---|
| Sample | Gleason Score | Staging | Risk status |
| Aggressive 1 | 8 | T1cNxMx | HR |
| Aggressive 2 | 8(5 + 3) | T3bN1M0 | VHR |
| Aggressive 3 | 8(4 + 4) | T2NxM1 | VHR |
| Aggressive 4 | 8(3 + 5) | T2NxM0 | HR |
| Aggressive 5 | 8(4 + 4) | T3NxM0 | VHR |
| Aggressive 6 | 9 | T2cNxMx | VHR |
| Aggressive 7 | 9(5 + 4) | T2NxM0 | VHR |
| Aggressive 8 | 9 | T2a NxM0 | VHR |
| Indolent 1 | 6 | T1cN0M0 | LR |
| Indolent 2 | 6 | T1cNxM0 | LR |
| Indolent 3 | 6 | T1cNxM0 | LR |
| Indolent 4 | 6 | T1aNxMx | LR |
| Normal 1 | N/A | N/A | Healthy |
| Normal 2 | N/A | N/A | Healthy |
| Normal 3 | N/A | N/A | Healthy |
| Normal 4 | N/A | N/A | Healthy |

TABLE 2
Clinical annotation for n = 16 patients screened for PCa.
The Gleason Score is the combined Gleason score obtained
through biopsy and histological examination.
Clinical tumor stage and cancer staging was based on
pathological examination. (All samples are serum, Male, Caucasian,
A = Aggressive, I = Indolent, N = Normal).

| Scano-miR | | | Patient | Gleason | PSA | |
|---|---|---|---|---|---|---|
| ID | Sample ID | Age | diagnosis | Score | ng/mL | Stage |
| Aggressive 7 | 161010S^{$} | 58 | Prostate | 9 | N/A | II |
| | | | Cancer | | | |
| Aggressive 2 | 161051S^{$} | 60 | Prostate | 8 | 8 | IV |
| | | | Cancer | | | |
| Aggressive 5 | 16712S^{$} | 76 | Prostate | 8 | 477 | III |
| | | | Cancer | | | |
| Aggressive 3 | 16906S^{$} | 54 | Prostate | 8 | 8.53 | IV |
| | | | Cancer | | | |
| Aggressive 8 | 11518552^{‡} | 66 | Prostate | 9 | 6.36 | N/A |
| | | | Cancer | | | |
| Aggressive 6 | 11518535^{‡} | 57 | Prostate | 9 | 6.8 | N/A |
| | | | Cancer | | | |
| Aggressive 4 | 16847S^{$} | 77 | Prostate | 8 | 45 | II |
| | | | Cancer | | | |
| Aggressive 1 | 11518542^{‡} | 63 | Prostate | 8 | N/A | N/A |
| | | | Cancer | | | |
| Indolent 1 | 11518536^{‡} | 71 | Prostate | 6 | N/A | N/A |
| | | | Cancer | | | |
| Indolent 4 | 11518558^{‡} | 53 | Prostate | 6 | N/A | N/A |
| | | | Cancer | | | |
| Indolent 2 | 11518537^{‡} | 62 | Prostate | 6 | N/A | N/A |
| | | | Cancer | | | |
| Indolent 3 | 11518539^{‡} | 73 | Prostate | 6 | N/A | N/A |
| | | | Cancer | | | |
| Normal 3 | D 2213S^{‡} | 63 | Normal | N/A | N/A | N/A |
| Normal 4 | D 2214S^{‡} | 66 | Normal | N/A | N/A | N/A |
| Normal 1 | D 2218S^{‡} | 65 | Normal | N/A | N/A | N/A |
| Normal 2 | D 2241S/Ac^{‡} | 60 | Normal | N/A | N/A | N/A |

^{$}Serum samples purchased from ProteoGenex, Inc., Culver City, CA.
^{‡}Serum samples purchased from ProMedDx, LLC, Norton, MA.

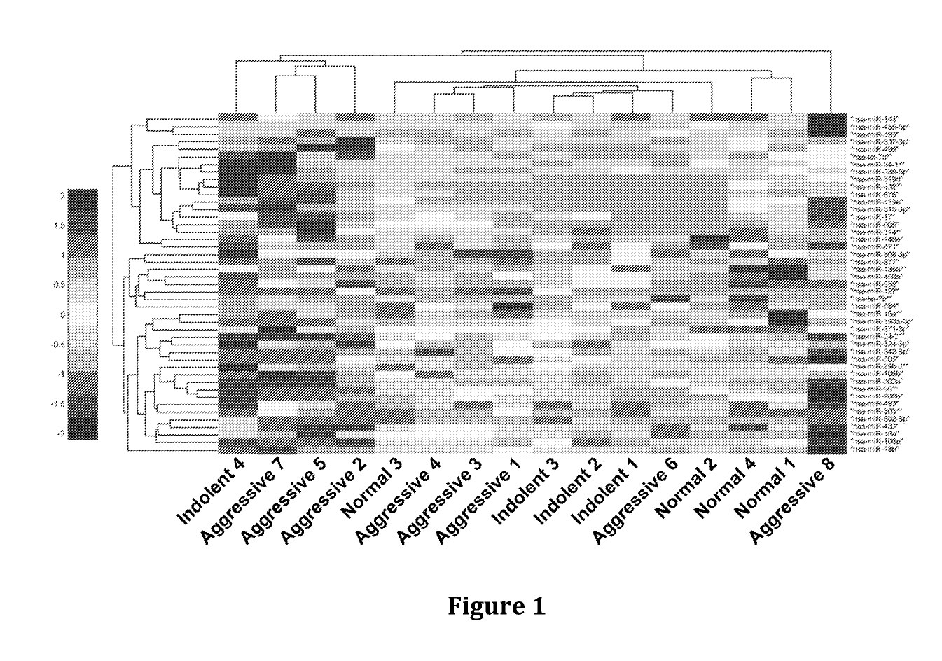

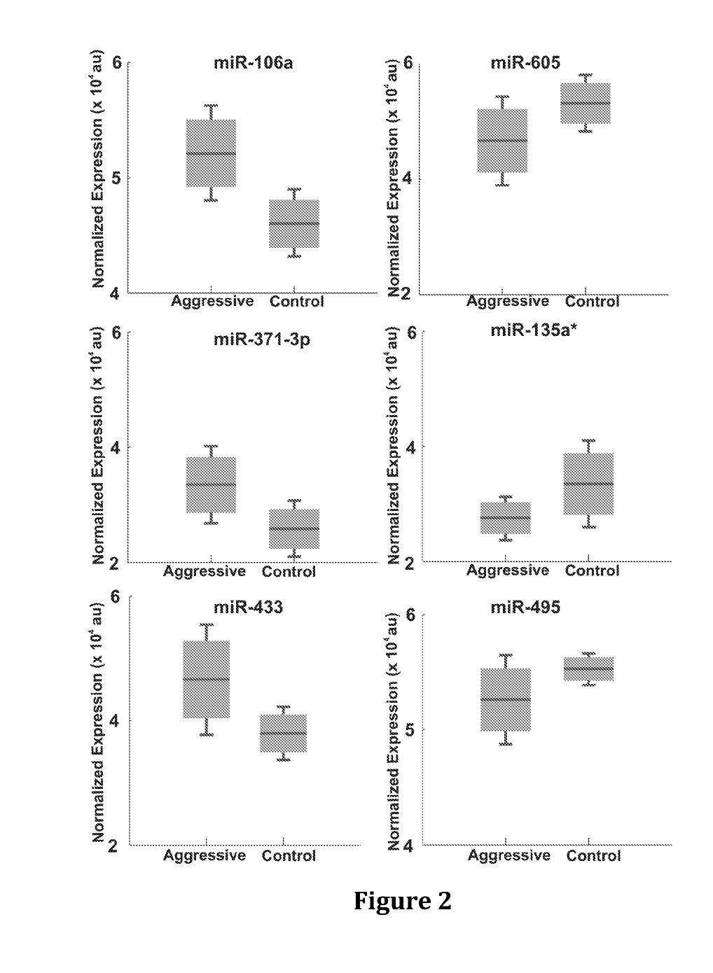

The comparison between the serum miRNA expression profiles of patients with a high Gleason score GS 8, HR and VHR, aggressive) to healthy individuals and to patients with a low Gleason score (GS 6, VLR or LR, controls) identified five exclusively expressed miRNAs (Table 3). Circulating miR-200c was the most frequently expressed marker (100%) in patients with a high Gleason score (n=8) and was below the detection limit of the Scano-miR assay in all other samples (GS 6 and healthy donors, n=8). Importantly, Scano-miR expression data analysis identified 58 miRNAs, consisting of 45 experimentally validated miRNAs and 13 predicted miRNAs (Table 4-5), which were co-expressed in all 16 samples. These co-expressed miRNAs were clustered based on their expression profiles using Pearson correlation in order to identify differentially expressed miRNAs (permutation t-test, p 0.05) (FIG. 1). Such hierarchical clustering identified 6 miRNAs with significant changes in their expression levels between aggressive and control samples (miR-605, miR-135a*, miR-495, miR-433, miR-371-3p, and miR-106a, with permutation t-test of p=0.0301, p=0.0319, p=0.0411, p=0.0115, p=0.0089, p=0.0017, respectively) (FIG. 2).

TABLE 3
List of miRNAs that are exclusively expressed in aggressive forms
of prostate cancer (PCa). Within n = 16 samples, multiple
microRNA species were detected solely in aggressive serum samples
that can serve as potential biomarkers. Frequency denotes the
number of times the microRNA was detected in the serum sample
divided by the number of aggressive samples (n = 8).

| Exclusively Expressed miRNAs in | |
|---|---|
| Aggressive (n = 8) vs. Control (n = 8) | Frequency |
| hsa-miR-200c | 100% |
| hsa-miR-219-2-3p | 75% |
| hsa-miR-337-5p | 50% |
| hsa-miR-331-3p | 50% |
| hsa-miR-409-3p | 50% |

TABLE 4
Expression data for co-expressed miRNAs in aggressive PCa. Each column denotes
the normalized final intensity value averaged between three probes for each
miRNA sequence screened. Enriched serum samples were taken from 8 aggressive
PCa patients (Gleason score >7) and screened using the ScanomiR platform.

| miRNA | Scano-A1 | Scano-A2 | Scano-A3 | Scano-A4 | Scano-A5 | Scano-A6 | Scano-A7 | Scano-A8 |
|---|---|---|---|---|---|---|---|---|
| let-7b* | 20549 | 28421 | 20549 | 20549 | 20549 | 31475 | 25790 | 20549 |
| let-7d* | 55720 | 29633 | 53004 | 54405 | 40877 | 54498 | 20549 | 47668 |
| miR-106a | 53004 | 56906 | 45530 | 49859 | 54405 | 46565 | 54958 | 56664 |
| miR-106b | 37522 | 35793 | 38247 | 32936 | 54498 | 35793 | 56092 | 46565 |
| miR-122 | 23263 | 32392 | 24777 | 25790 | 24777 | 38247 | 41639 | 39810 |
| miR-135a* | 24777 | 23263 | 29102 | 34772 | 29633 | 27231 | 24777 | 25790 |
| miR-144 | 45530 | 47668 | 46179 | 42281 | 43938 | 46179 | 43938 | 35793 |
| miR-148a | 56287 | 54958 | 54708 | 56287 | 47668 | 54132 | 54498 | 55600 |
| miR-15a* | 43938 | 54132 | 44905 | 48375 | 53947 | 47668 | 56906 | 49075 |
| miR-17 | 52681 | 49859 | 46565 | 43938 | 29102 | 50673 | 29102 | 29102 |
| miR-18a | 32392 | 32936 | 27231 | 29633 | 43147 | 20549 | 23263 | 48375 |
| miR-18b | 31475 | 27231 | 32936 | 28421 | 27231 | 25790 | 42281 | 44468 |
| miR-193a-3p | 48375 | 53947 | 49075 | 51709 | 56160 | 47081 | 56664 | 45530 |
| miR-200b | 25790 | 20549 | 30771 | 24777 | 41639 | 23263 | 44905 | 52681 |
| miR-214* | 47668 | 44905 | 47668 | 47668 | 23263 | 49859 | 40877 | 38247 |
| miR-24-1* | 56515 | 55173 | 56380 | 55988 | 54132 | 56906 | 45530 | 54405 |
| miR-24-2* | 46179 | 53004 | 33473 | 40877 | 55600 | 37522 | 56287 | 24777 |
| miR-29b-2* | 28421 | 29102 | 25790 | 29102 | 38247 | 33473 | 36417 | 33473 |
| miR-302a | 32936 | 30771 | 32392 | 35793 | 56380 | 39810 | 56515 | 56380 |
| miR-324-3p | 27231 | 37522 | 23263 | 23263 | 35793 | 29102 | 31475 | 23263 |
| miR-337-3p | 53504 | 36417 | 55720 | 56092 | 45530 | 55720 | 44468 | 47081 |
| miR-338-5p | 56906 | 56287 | 56664 | 56160 | 53504 | 56380 | 48375 | 56092 |
| miR-342-5p | 47081 | 54498 | 40877 | 36417 | 56515 | 43147 | 55988 | 39260 |
| miR-371-3p | 29102 | 33473 | 29633 | 27231 | 34772 | 32392 | 49859 | 29633 |
| miR-432* | 53947 | 40877 | 54132 | 53004 | 39260 | 55380 | 38247 | 46179 |
| miR-433 | 38247 | 54405 | 39260 | 41639 | 55173 | 32936 | 54405 | 56287 |
| miR-450a | 39810 | 42281 | 36417 | 37522 | 37522 | 39260 | 37522 | 42281 |
| miR-455-5p | 56092 | 55988 | 56092 | 55600 | 56287 | 53947 | 56160 | 43938 |
| miR-493 | 46565 | 41639 | 53504 | 46565 | 51709 | 44468 | 49075 | 56160 |
| miR-495 | 54958 | 47081 | 55988 | 55380 | 47081 | 54958 | 52681 | 54132 |
| miR-502-5p | 44905 | 55380 | 43938 | 45530 | 56092 | 43938 | 56380 | 56515 |
| miR-505 | 40877 | 48375 | 42281 | 44905 | 55988 | 45530 | 54708 | 37522 |
| miR-505* | 49859 | 56664 | 44468 | 46179 | 55380 | 48375 | 51709 | 56906 |
| miR-508-3p | 36417 | 31475 | 37522 | 33473 | 33473 | 28421 | 32392 | 31475 |
| miR-515-3p | 54132 | 46565 | 53947 | 53504 | 39810 | 56092 | 30771 | 32936 |
| miR-519d | 54405 | 49075 | 54405 | 54958 | 44468 | 56515 | 43147 | 50673 |
| miR-519e | 55600 | 39810 | 55380 | 54708 | 28421 | 56160 | 29633 | 30771 |
| miR-558 | 41639 | 56380 | 39810 | 44468 | 52681 | 40877 | 47081 | 55380 |
| miR-584 | 33473 | 54708 | 51709 | 49075 | 53004 | 49075 | 54132 | 53947 |
| miR-595 | 56664 | 50673 | 50673 | 56664 | 48375 | 52681 | 55173 | 40877 |
| miR-605 | 55173 | 45530 | 49859 | 50673 | 36417 | 56287 | 39260 | 41639 |
| miR-675 | 54708 | 43938 | 54958 | 52681 | 31475 | 55988 | 33473 | 44905 |
| miR-871 | 56160 | 56160 | 55600 | 56906 | 54958 | 54405 | 55380 | 53504 |
| miR-877 | 50673 | 52681 | 55173 | 55173 | 56664 | 51709 | 55720 | 49859 |
| miR-96* | 34772 | 25790 | 34772 | 31475 | 44905 | 29633 | 47668 | 55173 |
| IVGN-novel- | 35793 | 34772 | 48375 | 47081 | 49075 | 41639 | 50673 | 54498 |
| miR_3446 | | | | | | | | |
| IVGN-novel- | 42281 | 24777 | 35793 | 39810 | 42281 | 36417 | 53004 | 55720 |
| miR_3458 | | | | | | | | |
| IVGN-novel- | 43147 | 39260 | 43147 | 43147 | 46565 | 44905 | 35793 | 43147 |
| miR_3513 | | | | | | | | |
| IVGN-novel- | 30771 | 43147 | 31475 | 30771 | 56906 | 24777 | 55600 | 27231 |
| miR_3515 | | | | | | | | |
| IVGN-novel- | 55380 | 38247 | 54498 | 54498 | 32392 | 53004 | 34772 | 32392 |
| miR_3516 | | | | | | | | |
| IVGN-novel- | 44468 | 44468 | 47081 | 39260 | 30771 | 42281 | 32936 | 36417 |
| miR_3517 | | | | | | | | |
| IVGN-novel- | 55988 | 53504 | 56160 | 56380 | 50673 | 54708 | 46565 | 51709 |
| miR_3575 | | | | | | | | |
| IVGN-novel- | 54498 | 55720 | 56906 | 55720 | 46179 | 55600 | 53504 | 53004 |
| miR_3582 | | | | | | | | |
| IVGN-novel- | 39260 | 46179 | 41639 | 32392 | 32936 | 30771 | 39810 | 34772 |
| miR_3645 | | | | | | | | |
| IVGN-novel- | 51709 | 56515 | 52681 | 54132 | 54708 | 53504 | 27231 | 55988 |
| miR_3674 | | | | | | | | |
| IVGN-novel- | 56380 | 55600 | 56287 | 56515 | 55720 | 56664 | 53947 | 54958 |
| miR_3683 | | | | | | | | |
| IVGN-novel- | 29633 | 51709 | 28421 | 38247 | 25790 | 34772 | 28421 | 28421 |
| miR_3696 | | | | | | | | |
| IVGN-novel- | 49075 | 56092 | 56515 | 53947 | 49859 | 55173 | 46179 | 54708 |
| miR_3702 | | | | | | | | |

TABLE 5
Expression data for co-expressed miRNAs in normal and indolent PCa. Each column denotes the normalized
final intensity value averaged between three probes for each miRNA sequence screened. 4 indolent (Gleason
score = 6) and 4 normal enriched serum samples were screened using the Scano-miR platform.

| miRNA | Scano-I5 | Scano-I2 | Scano-I3 | Scano-I4 | Scano-N1 | Scano-N2 | Scano-N3 | Scano-N4 |
|---|---|---|---|---|---|---|---|---|
| let-7b* | 25790 | 23263 | 20549 | 20549 | 28421 | 27231 | 25790 | 32936 |
| let-7d* | 56092 | 55600 | 53504 | 25790 | 48375 | 51709 | 49859 | 55380 |
| miR-106a | 45530 | 43147 | 49075 | 40877 | 49075 | 48375 | 46565 | 46179 |
| miR-106b | 38247 | 36417 | 38247 | 52681 | 44468 | 40877 | 42281 | 42281 |
| miR-122 | 36417 | 38247 | 34772 | 39810 | 39260 | 28421 | 24777 | 37522 |
| miR-135a* | 23263 | 31475 | 29633 | 29102 | 43938 | 31475 | 32392 | 45530 |
| miR-144 | 47668 | 44905 | 46179 | 47081 | 40877 | 39260 | 41639 | 38247 |
| miR-148a | 52681 | 56092 | 53947 | 49859 | 55988 | 47081 | 54132 | 47668 |
| miR-15a* | 47081 | 49075 | 48375 | 56160 | 35793 | 53504 | 56515 | 55720 |
| miR-17 | 48375 | 43938 | 47668 | 42281 | 45530 | 50673 | 45530 | 39810 |
| miR-18a | 32392 | 24777 | 27231 | 30771 | 23263 | 25790 | 28421 | 23263 |
| miR-18b | 28421 | 35793 | 35793 | 23263 | 24777 | 32392 | 32936 | 27231 |
| miR-193a-3p | 49859 | 52681 | 49859 | 56906 | 38247 | 49859 | 54708 | 50673 |
| miR-200b | 20549 | 25790 | 25790 | 46179 | 29102 | 23263 | 20549 | 29102 |
| miR-214* | 50673 | 53504 | 51709 | 38247 | 41639 | 53004 | 47081 | 34772 |
| miR-24-1* | 56515 | 56380 | 56664 | 43147 | 55600 | 54708 | 54958 | 56287 |
| miR-24-2* | 35793 | 37522 | 41639 | 56664 | 36417 | 41639 | 38247 | 39260 |
| miR-29b-2* | 29102 | 29102 | 32936 | 55173 | 29633 | 29633 | 23263 | 29633 |
| miR-302a | 32936 | 34772 | 36417 | 55988 | 34772 | 32936 | 36417 | 33473 |
| miR-324-3p | 30771 | 20549 | 23263 | 43938 | 25790 | 29102 | 27231 | 24777 |
| miR-337-3p | 53004 | 53004 | 53004 | 55720 | 54132 | 54498 | 49075 | 51709 |
| miR-338-5p | 55600 | 55720 | 56515 | 44905 | 54958 | 56515 | 56092 | 55600 |
| miR-342-5p | 46179 | 42281 | 47081 | 55600 | 50673 | 46179 | 54498 | 54405 |
| miR-371-3p | 24777 | 28421 | 29102 | 31475 | 20549 | 20549 | 29633 | 20549 |
| miR-432* | 54132 | 54498 | 54958 | 28421 | 42281 | 56380 | 52681 | 46565 |
| miR-433 | 37522 | 39260 | 37522 | 45530 | 37522 | 38247 | 39810 | 31475 |
| miR-450a | 40877 | 41639 | 39260 | 29633 | 53004 | 45530 | 37522 | 47081 |
| miR-455-5p | 53947 | 56515 | 54708 | 56287 | 56906 | 55988 | 56664 | 56092 |
| miR-493 | 43938 | 46179 | 42281 | 56092 | 52681 | 46565 | 44905 | 54498 |
| miR-495 | 56664 | 56664 | 56380 | 54405 | 56160 | 55380 | 53947 | 53504 |
| miR-502-5p | 44905 | 47081 | 43147 | 53504 | 54708 | 44905 | 56906 | 44468 |
| miR-505 | 49075 | 46565 | 44468 | 54958 | 56092 | 43938 | 43938 | 53004 |
| miR-505* | 44468 | 44468 | 44905 | 56380 | 55720 | 47668 | 43147 | 55173 |
| miR-508-3p | 31475 | 29633 | 32392 | 24777 | 31475 | 30771 | 31475 | 36417 |
| miR-515-3p | 55988 | 54708 | 56160 | 32392 | 53504 | 56664 | 51709 | 49859 |
| miR-519d | 56287 | 54958 | 55720 | 33473 | 47668 | 56287 | 53504 | 48375 |
| miR-519e | 56160 | 54132 | 56092 | 35793 | 47081 | 54958 | 46179 | 43938 |
| miR-558 | 46565 | 45530 | 43938 | 36417 | 54405 | 43147 | 40877 | 55988 |
| miR-584 | 43147 | 49859 | 45530 | 44468 | 46565 | 56092 | 56160 | 56664 |
| miR-595 | 54405 | 50673 | 54405 | 55380 | 56664 | 55173 | 55380 | 53947 |
| miR-605 | 56380 | 55380 | 55988 | 56515 | 53947 | 56906 | 47668 | 41639 |
| miR-675 | 54708 | 54405 | 56287 | 27231 | 39810 | 56160 | 48375 | 40877 |
| miR-871 | 55720 | 56906 | 55600 | 54708 | 56287 | 54132 | 55988 | 54958 |
| miR-877 | 53504 | 51709 | 50673 | 51709 | 56380 | 52681 | 55720 | 54132 |
| miR-96* | 29633 | 32936 | 33473 | 47668 | 30771 | 35793 | 35793 | 35793 |
| IVGN-novel- | 42281 | 47668 | 46565 | 50673 | 46179 | 39810 | 56287 | 56380 |
| miR_3446 | | | | | | | | |
| IVGN-novel- | 33473 | 30771 | 30771 | 54498 | 32936 | 34772 | 39260 | 28421 |
| miR_3458 | | | | | | | | |
| IVGN-novel- | 39810 | 40877 | 39810 | 37522 | 33473 | 37522 | 30771 | 32392 |
| miR_3513 | | | | | | | | |
| IVGN-novel- | 39260 | 32392 | 31475 | 53004 | 32392 | 24777 | 34772 | 30771 |
| miR_3515 | | | | | | | | |
| IVGN-novel- | 55380 | 53947 | 55380 | 32936 | 49859 | 55720 | 50673 | 49075 |
| miR_3516 | | | | | | | | |
| IVGN-novel- | 41639 | 48375 | 40877 | 41639 | 44905 | 49075 | 44468 | 44905 |
| miR_3517 | | | | | | | | |
| IVGN-novel- | 54958 | 55988 | 55173 | 54132 | 54498 | 42281 | 53004 | 52681 |
| miR_3575 | | | | | | | | |
| IVGN-novel- | 55173 | 55173 | 54498 | 46565 | 55173 | 44468 | 55600 | 56160 |
| miR_3582 | | | | | | | | |
| IVGN-novel- | 27231 | 33473 | 24777 | 34772 | 27231 | 36417 | 33473 | 25790 |
| miR_3645 | | | | | | | | |
| IVGN-novel- | 51709 | 39810 | 52681 | 49075 | 56515 | 55600 | 55173 | 56906 |
| miR_3674 | | | | | | | | |
| IVGN-novel- | 56906 | 56287 | 56906 | 53947 | 55380 | 53947 | 54405 | 54708 |
| miR_3683 | | | | | | | | |
| IVGN-novel- | 34772 | 27231 | 28421 | 39260 | 43147 | 33473 | 29102 | 43147 |
| miR_3696 | | | | | | | | |
| IVGN-novel- | 54498 | 56160 | 54132 | 48375 | 51709 | 54405 | 56380 | 56515 |
| miR_3702 | | | | | | | | |

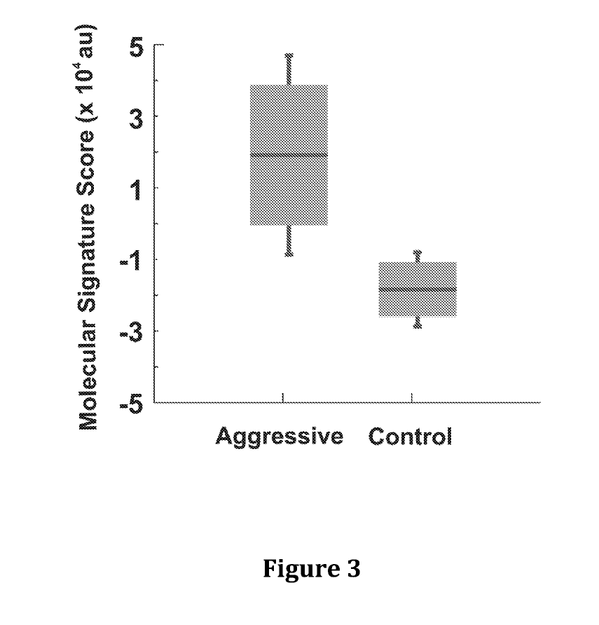

Despite the identification of differentially expressed miRNAs, single biomarkers may not be accurate diagnostics for aggressive PCa. For that reason, the molecular signature score was calculated for the differentially expressed miRNAs to distinguish aggressive PCa from control samples using a published mathematical formula.³² The molecular signature analysis revealed that the diagnostic reliability was increased significantly (p=0.0036) upon combining the differentially expressed miRNAs (FIG. 3). However, the molecular signature score was able to detect 50% of the aggressive PCa samples, which suggests either the identified molecular signature could not be used as a reliable indicator for the detection of aggressive PCa, or there might be more intermediate grades of PCa that were not distinguished using the Gleason sum of the prostatic needle biopsy specimens. To address these questions, we performed correlation studies to the clinical pathology of PCa instead, and validation studies using blinded patients.

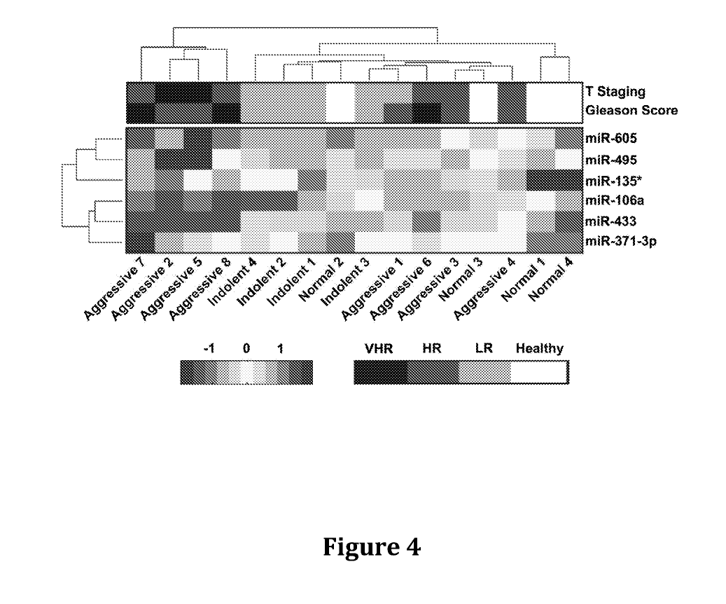

Current diagnostic criteria exhibit a rate of misclassification of up to 20% when it comes to discriminating patients with slow progressive PCa from patients at high risk of developing a more aggressive cancer.³³ For that reason, we investigated the correlation between the molecular signature and the clinical pathology of tumors to differences in the rate of cancer progression. To examine such a correlation, patients with GS >6 were grouped into either very high-risk (VHR) aggressive or high-risk (HR) aggressive PCas based upon clinicopathologic features following the 2015 NCCN Guidelines for Prostate Cancer (Version 1.2015). Using such criteria, six patients were identified as having VHR cancer that progressed into a highly aggressive PCa (GS 9, metastasis, and/or clinical stage T3), and two patients with HR PCa (GS<9 and clinical stage <T3) (Table 1). Hierarchical clustering of the molecular signature identified a subclass of four patients, where the PCa of these patients were classified as VHR (FIG. 4). Moreover, we calculated the correlation of the molecular signature and individual miRNAs to the VHR cancer using the Kaplan-Meier and Wilcoxon rank sum tests.³⁴′³⁵ The results demonstrate a significant correlation of the identified molecular signature to the clinical pathology of these patients (p=0.041) (Table 6). In contrast, not all differentially expressed miRNAs show high correlations when examined individually (3 miRNAs with p 0.05), which support the notion that individual biomarkers are not indicative of disease states.

TABLE 6
Aggregate of six miRNAs showed significant correlation
to highly aggressive PCa. The combined signature intensity
is correlated to the degree of PCa aggressiveness (n =
16). Correlation between miRNA expression and patient risk
was analyzed using the Wilcoxon rank-sum test.

| Biomarker | Correlation to risk status^{¶} |
|---|---|
| Molecular Signature | * |
| hsa-mir-605 | * |
| hsa-mir-135a* | — |
| hsa-mir-495 | * |
| hsa-mir-433 | — |
| hsa-mir-371-3p | * |
| hsa-mir-106a | Trend |

^{¶}Correlation p-value; p < 0.1 “trend”, p < 0.05 “*”, No correlation “—”.

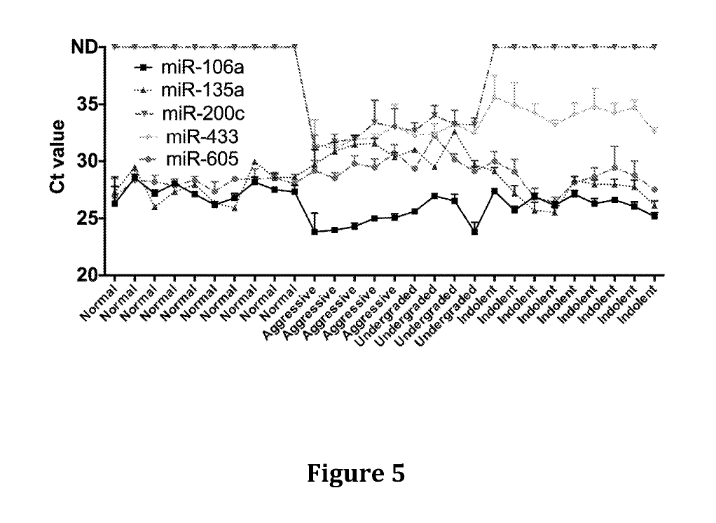

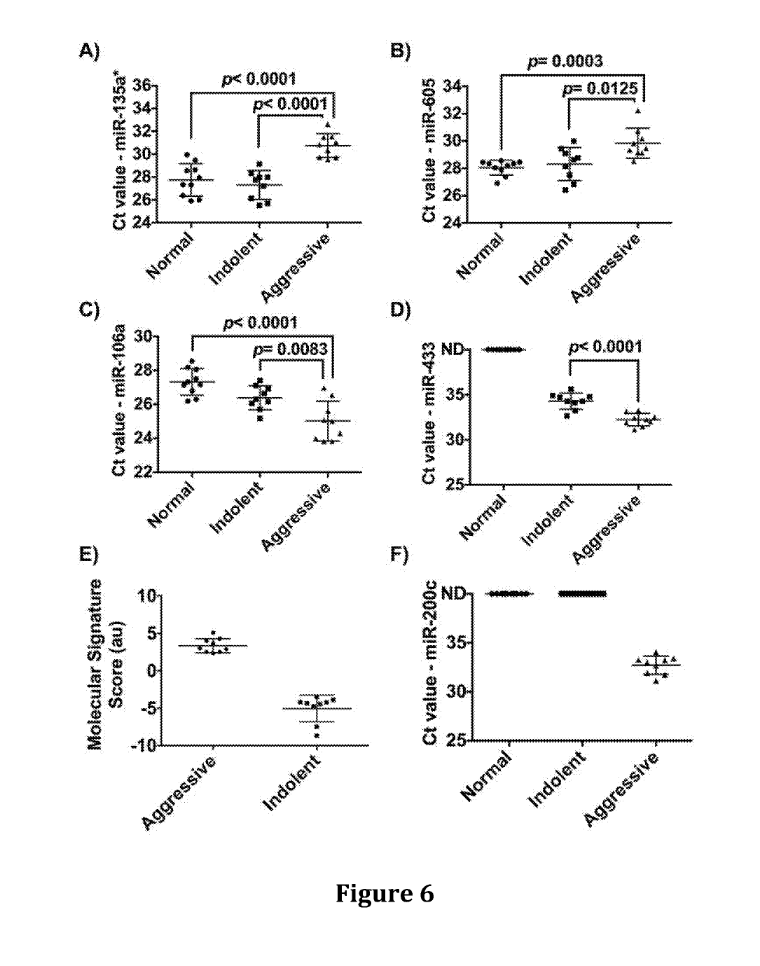

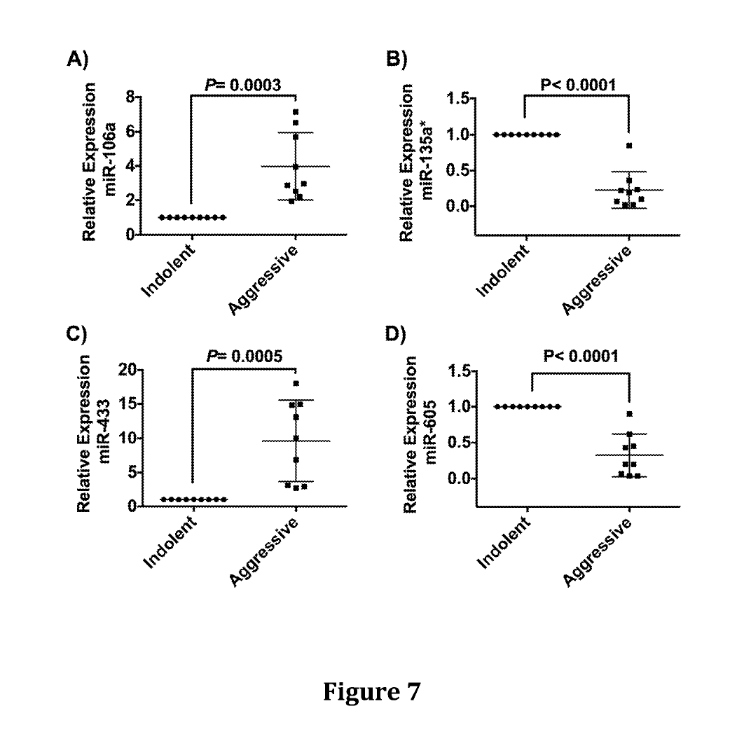

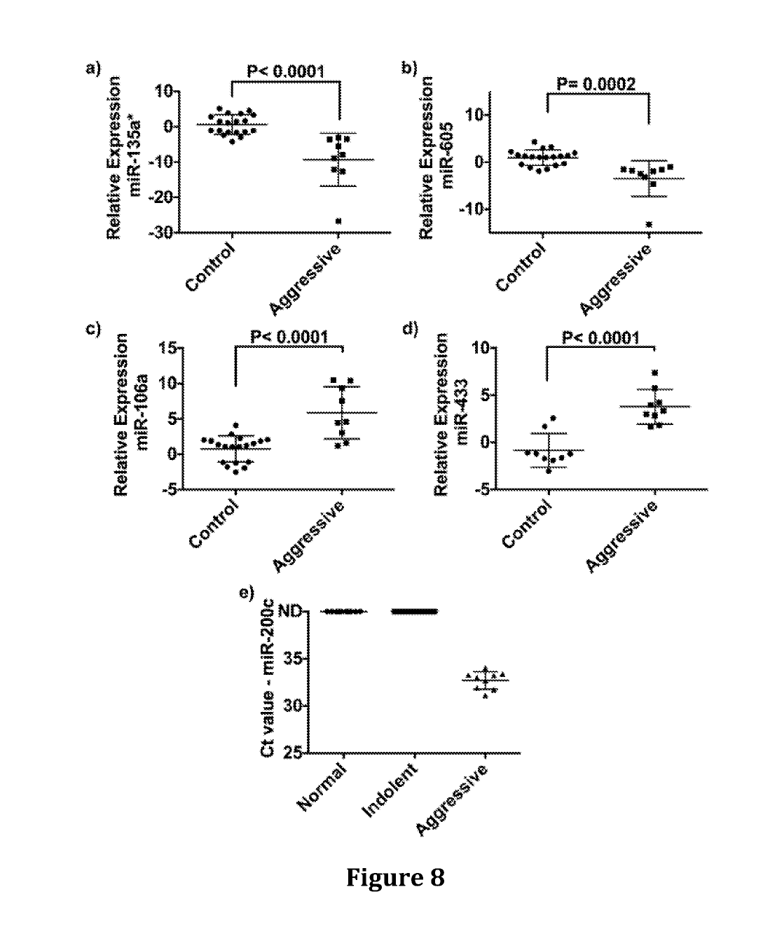

In order to validate the reliability of the identified circulating miRNAs as diagnostic biomarkers, we obtained additional clinical serum samples from de-identified patients (highly aggressive PCa, indolent, and healthy donors, with sample size n=9, n=9, and n=10, respectively) and performed the blind test. The clinical annotation data for these samples are included in Table 7. Five miRNAs were successfully detected using qRT-PCR (miR-200c, miR-605, miR-135a*, miR-433, and miR-106a) that exhibited the same expression profiles as in our Scano-miR studies (FIG. 5-6). Four of these miRNAs were differentially expressed in highly aggressive and undergraded PCas relative to indolent PCas (FIG. 6A-D and FIG. 8) with fold changes >1.5 (FIG. 7 and FIG. 8). The molecular signature score was calculated and it was found that these four miRNAs significantly distinguished clinically significant PCa from indolent PCa (FIG. 6E). miR-433 was differentially expressed in highly aggressive versus indolent PCa serum samples (p<0.0001), but was not detected in normal serum samples (FIG. 6D). In addition, miR-200c was only detected in serum samples from patients with highly aggressive PCa (FIG. 6F). The data show that these miRNAs can be used as non-invasive biomarkers to distinguish between patients with aggressive and indolent forms of PCa.

TABLE 7
Clinical annotation for n = 28 blinded donors screened for PCa.
The Gleason sum is the combined Gleason score obtained
through histological examination of both prostatic needle
biopsy and radical prostatectomy (RP). Clinical tumor
stage and cancer staging were based on pathological
examination. (All samples are serum, Male, Caucasian,
A = Aggressive, I = Indolent, N = Normal).
N/A = Not available. VHR—very high risk,
HR—high risk, LR—low risk. Patients were categorized based
on the 2015 NCCN Guidelines for Prostate Cancer
(Version 1.2015)

| | | | | First | | |
|---|---|---|---|---|---|---|
| | | | | Biopsy | RP | Patho- |
| | | | Risk | Total | Total | logical |
| Sample ID | Age | Category | status | Gleason | Gleason | Stage |
| 415162^{$} | 49 | Aggres- | VHR | 5 + 4 | 5 + 4 | T3a |
| | | sive | | | | |
| 415163^{$} | 67 | Aggres- | VHR | 4 + 5 | 4 + 5 | T3b |
| | | sive | | | | |
| 415164^{$} | 73 | Aggres- | VHR | 4 + 5 | 4 + 5 | T3b |
| | | sive | | | | |
| 415165^{$} | 76 | Aggres- | VHR | 4 + 4 | 5 + 3 | T3b |
| | | sive | | | | |
| 62985^{$} | 60 | Aggres- | VHR | 4 + 5 | 4 + 5 | T3a |
| | | sive | | | | |
| 33220^{$} | 66 | Under- | LR | 3 + 3 | 4 + 4 | T3b |
| | | graded | | | | |
| 415173^{$} | 76 | Under- | LR | 3 + 3 | 4 + 4 | T3b |
| | | graded | | | | |
| 41145^{$} | 69 | Under- | LR | 3 + 3 | 3 + 5 | T3a |
| | | graded | | | | |
| 415172^{$} | 74 | Under- | LR | 3 + 3 | 5 + 4 | T3a |
| | | graded | | | | |
| 36957^{$} | 67 | Indolent | LR | 3 + 3 | 3 + 3 | T2c |
| 36936^{$} | 71 | Indolent | LR | 3 + 3 | 3 + 3 | T2c |
| 38123^{$} | 65 | Indolent | LR | 3 + 3 | 3 + 3 | T2a |
| 415166^{$} | 79 | Indolent | LR | 3 + 3 | 3 + 3 | T3a |
| 415167^{$} | 76 | Indolent | LR | 3 + 3 | 3 + 3 | T2a |
| 415168^{$} | 66 | Indolent | LR | 3 + 3 | 3 + 3 | T2a |
| 415169^{$} | 67 | Indolent | LR | 3 + 3 | 3 + 3 | T2c |
| 415170^{$} | 59 | Indolent | LR | 3 + 3 | 3 + 3 | T2b |
| 415171^{$} | 77 | Indolent | LR | 3 + 3 | 3 + 3 | T2c |
| BRH980191^{‡} | 50 | Normal | Healthy | N/A | N/A | N/A |
| BRH991717^{‡} | 59 | Normal | Healthy | N/A | N/A | N/A |
| BRH991716^{‡} | 50 | Normal | Healthy | N/A | N/A | N/A |
| BRH991715^{‡} | 54 | Normal | Healthy | N/A | N/A | N/A |
| BRH991714^{‡} | 59 | Normal | Healthy | N/A | N/A | N/A |
| BRH991712^{‡} | 50 | Normal | Healthy | N/A | N/A | N/A |
| BRH991711^{‡} | 50 | Normal | Healthy | N/A | N/A | N/A |
| BRH991710^{‡} | 65 | Normal | Healthy | N/A | N/A | N/A |
| BRH991709^{‡} | 54 | Normal | Healthy | N/A | N/A | N/A |
| BRH991708^{‡} | 57 | Normal | Healthy | N/A | N/A | N/A |

^{$}Serum samples obtained from the NU Prostate SPORE serum repository, Chicago, IL.
^{‡}Serum samples purchased from BioreclamationIVT, Baltimore, MD.

Seven miRNAs were identified using the Scano-miR assay (miR-605, miR-135a*, miR-495, miR-433, miR-371-3p, miR-200c, and miR-106a). The discovery and identification of novel biomarkers for PCa diagnosis is necessary in order to address the inaccuracies of PSA screening and Gleason scoring based solely on prostatic needle biopsy specimens. As such, the accuracy of the miRNA-based molecular signature score disclosed herein was compared to both prostatic needle biopsy and radical prostatectomy Gleason scoring in differentiating between aggressive and indolent forms of PCa. The results show that the miRNAs identified by the Scano-miR bioassay are at least 94% accurate in differentiating between aggressive versus indolent PCa, while the prostatic needle biopsy Gleason grading technique is only 77% accurate (Table 8). Non-invasive profiling of these miRNA biomarkers may enable rapid diagnosis and accurate prediction of PCa without unnecessary surgical or treatment regimens.

TABLE 8
The miRNAs identified by the Scano-miR bioassay are at least 94%
accurate in differentiating between aggressive versus indolent PCa.
Accuracy of Gleason sum from prostatic needle biopsy and miRNA-based
molecular signature score vs. radical prostatectomy (RP).

| | | # of patients |
|---|---|---|
| | | Aggressive (n = 9) |
| Biomarker | Accuracy % | vs. indolent (n = 9) |
| RP Gleason score | 100.00 | 18/18 |
| Biopsy Gleason score | 77.78 | 14/18 |
| miR-200c | 100.00 | 18/18 |
| miR-433 | 100.00 | 18/18 |
| miR-135a* | 94.44 | 17/18 |
| miR-106a | 100 | 18/18 |
| miR-605 | 94.44 | 17/18 |

In addition, the majority of the identified miRNAs were linked previously to the pathogenesis of the prostate cancer either as oncogenes or tumor suppressors. For example, circulating miR-200c in plasma can be used as a marker to distinguish localized PCa from metastatic castration resistant PCa.³⁶ miR-106a and miR-135a were significantly upregulated in PCa, while a single nucleotide polymorphism in miR-605 was found to correlate with the biochemical recurrence of PCa.³⁷⁻³⁹ However, the selected miRNAs panel (miR-200c, miR-605, miR-135a*, miR-433, and miR-106a) was not identified previously to have predictive value for the management of PCa.

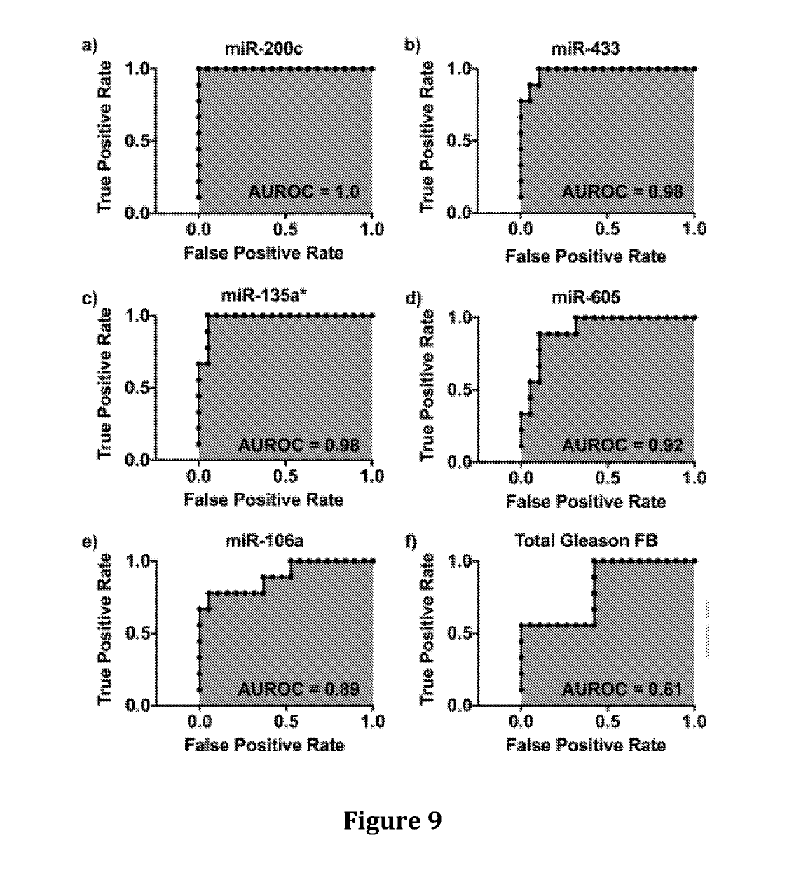

One of the potential applications of the miRNA score is to accurately risk stratify patients with biopsy-detected PCa. Thus, the area under the curve (AUC) of the miRNA score was compared to the gold standard—the biopsy identified Gleason score—for predicting aggressive compared to indolent cancer. Receiver operating characteristic (ROC) analyses showed that the miRNAs identified by the Scano-miR bioassay exhibit very high diagnostic capabilities in differentiating between VHR aggressive PCa versus controls with a ROC of 1.0, 0.98, 0.98, 0.92, and 0.89 for miR-200c, miR-433, miR-135a*, miR-605, and miR-106a, respectively (FIG. 9a-9e). The prostatic needle biopsy Gleason grading showed the lowest diagnostic capability with an ROC of 0.81 (FIG. 9f).

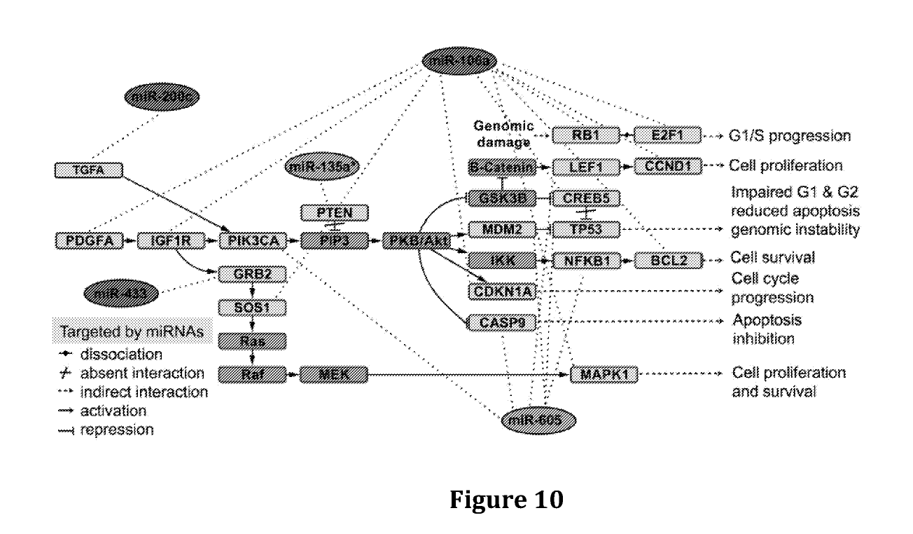

Mapping the validated miRNAs to PCa pathways is important toward understanding their significance in PCa progression. In silico analyses generated a total of 42 candidate pathways (Table 9) from which five common pathways are targeted by the validated miRNA biomarker panel. The identified pathways are primarily involved in cancer progression (including PCa) and PI3K-Akt signaling (Table 10). The PI3K-Akt signaling pathway was found to be among the top common candidate pathways, which is a major driver of PCa growth in advanced cancer stages. Additionally, genes that are known to be involved in PCa progression were significant targets of the validated miRNAs (corrected p-value threshold of <0.05; FIG. 10). The results show that the validated miRNAs target different genes within the same candidate pathways involved in the transition from localized PCa to metastatic PCa.

TABLE 9
Enriched KEGG pathway analysis. Table showing biological pathways
associated with validated miRNA PCa biomarkers. Common pathways
associated with all five miRNAs are shown in bold font.

| | | # of | # of |
|---|---|---|---|
| KEGG pathway | p-value | genes | miRNA |
| TGF-beta signaling pathway (hsa04350) | 2.55E−11 | 19 | 2 |
| Endocytosis (hsa04144) | 3.28E−09 | 35 | 3 |
| Hepatitis B (hsa05161) | 2.51E−08 | 24 | 4 |
| ErbB signaling pathway (hsa04012) | 1.34E−06 | 18 | 4 |
| Colorectal cancer (hsa05210) | 1.67E−06 | 14 | 2 |
| Chronic myeloid leukemia (hsa05220) | 1.73E−06 | 16 | 4 |
| Pathways in cancer | 2.94E−06 | 44 | 5 |
| (hsa05200) | | | |
| Acute myeloid leukemia (hsa05221) | 5.51E−06 | 13 | 3 |
| PI3K-Akt signaling pathway | 5.51E−06 | 44 | 5 |
| (hsa04151) | | | |
| Prostate cancer (hsa05215) | 1.06E−05 | 17 | 5 |
| Pancreatic cancer (hsa05212) | 2.34E−05 | 15 | 3 |
| Hepatitis C (hsa05160) | 4.23E−05 | 21 | 4 |
| Neurotrophin signaling pathway | 4.59E−05 | 20 | 4 |
| (hsa04722) | | | |
| Insulin signaling pathway (hsa04910) | 1.38E−04 | 21 | 4 |
| Protein processing in endoplasmic | 6.11E−04 | 25 | 3 |
| reticulum(hsa04141) | | | |
| Dorso-ventral axis formation (hsa04320) | 1.23E−03 | 6 | 3 |
| Regulation of autophagy (hsa04140) | 1.45E−03 | 7 | 2 |
| Axon guidance (hsa04360) | 1.47E−03 | 20 | 2 |
| Gap junction (hsa04540) | 1.49E−03 | 12 | 4 |
| Endometrial cancer (hsa05213) | 1.93E−03 | 10 | 4 |
| Focal adhesion (hsa04510) | 1.93E−03 | 26 | 5 |
| Taurine and hypotaurine metabolism | 2.21E−03 | 3 | 3 |
| (hsa00430) | | | |
| Pantothenate and CoA biosynthesis | 2.23E−03 | 5 | 2 |
| (hsa00770) | | | |
| p53 signaling pathway (hsa04115) | 2.23E−03 | 12 | 3 |
| Maturity onset diabetes of the young | 2.77E−03 | 5 | 3 |
| (hsa04950) | | | |
| Spliceosome (hsa03040) | 3.45E−03 | 18 | 4 |
| MAPK signaling pathway (hsa04010) | 3.65E−03 | 31 | 4 |
| Ubiquitin mediated proteolysis | 5.51E−03 | 19 | 3 |
| (hsa04120) | | | |
| Wnt signaling pathway (hsa04310) | 5.53E−03 | 22 | 3 |
| Non-small cell lung cancer (hsa05223) | 8.77E−03 | 9 | 4 |
| Shigellosis (hsa05131) | 1.28E−02 | 10 | 2 |
| ARVC (hsa05412) | 1.31E−02 | 10 | 2 |
| Glioma (hsa05214) | 1.36E−02 | 11 | 5 |
| beta-Alanine metabolism (hsa00410) | 1.84E−02 | 6 | 2 |
| Circadian rhythm (hsa04710) | 1.84E−02 | 6 | 2 |
| Chagas disease (American | 1.84E−02 | 14 | 3 |
| trypanosomiasis)(hsa05142) | | | |
| Thyroid cancer (hsa05216) | 2.90E−02 | 6 | 2 |
| mRNA surveillance pathway (hsa03015) | 2.96E−02 | 12 | 3 |
| Small cell lung cancer (hsa05222) | 3.19E−02 | 11 | 3 |
| Melanoma (hsa05218) | 3.45E−02 | 10 | 4 |
| Valine, leucine and isoleucine | 3.82E−02 | 1 | 1 |
| biosynthesis(hsa00290) | | | |
| Epithelial cell signaling in H. pylori | 4.27E−02 | 9 | 2 |
| infection (hsa05120) | | | |

TABLE 10
Common KEGG Pathways. Table showing biological pathways
shared with the five validated miRNA PCa biomarkers.

| | | # of | # of |
|---|---|---|---|
| KEGG Pathway | p-value | genes | miRNA |
| Pathways in cancer (hsa05200) | 2.94E−06 | 44 | 5 |
| PI3K-Akt signaling pathway | 5.51E−06 | 44 | 5 |
| (hsa04151) | | | |
| Prostate cancer (hsa05215) | 1.06E−05 | 17 | 5 |
| Focal adhesion (hsa04510) | 1.93E−03 | 26 | 5 |
| Glioma (hsa05214) | 1.36E−02 | 11 | 5 |

### Discussion

Risk stratified treatment of PCa is critically dependent on staging through PSA, physical exam and tissue biopsy. To address the inherent gaps in cancer staging, the Scano-miR profiling platform was successfully applied and validated and ultimately identified a unique panel of miRNA biomarkers associated with different grades of PCa.

The miRNA biomarker panel was discovered and validated by investigation of the serum miRNA profiles from two experimental sample sets. The first set was profiled using the Scano-miR bioassay in order to identify differentially expressed miRNAs specific to VHR PCa samples that were previously clinically graded based upon Gleason biopsy scoring. A blinded qRT-PCR study was then performed on the second sample set which served to validate the identified miRNA biomarkers in patient samples with known pathological grading. For example, while individual miRNA biomarkers such as miR-433 and miR-135a* did not fully agree with the clinical grading of PCa, known pathological grading of the blinded qRT-PCR study validated the significant diagnostic capabilities of the identified miRNA biomarkers including circulating miR-433 and miR-135a*. The molecular signature generated from the validated miRNAs enabled the accurate distinction between patients with indolent or aggressive forms of PCa at rates higher than typical prostatic needle biopsy Gleason scoring. This miRNA biomarker panel represents a simple tool for the diagnosis of PCa without the need for surgical intervention.

The majority of the identified miRNAs were linked previously to the pathogenesis of PCa either as oncogenes or tumor suppressors. Circulating miR-200c in plasma can be used as a marker to distinguish localized PCa from metastatic castration resistant PCa [WatahikiA, et al. (2013) Int. J. Mol. Sci. 14(4):7757-7770]. miR-106a was significantly dysregulated in PCa, while a single nucleotide polymorphism in miR-605 was found to cor-relate with the biochemical recurrence of PCa [Volinia S, et al. (2006) Proc. Natl. Acad. Sci. USA 103(7):2257-2261; Huang S P, et al. (2014) Int. J. Cancer 135(11): 2661-2667]. However, circulating miR-433 and miR-135a* have not been linked to PCa previously, and the selected miRNA panel (miR-200c, miR-605, miR-135a*, miR-433, and miR-106a) has not been proposed to have a predictive value for the management of PCa.

Identifying genetic clues to the molecular basis of PCa growth is a major challenge since the number of mutated genes is often higher than the actual mutations that drive cancer. The present analysis with the selected miRNA panel in the PCa pathway suggested a list of target genes {PTEN, PI3K, TP53, RBI, MDM2, TGFA, NFKB1, CASP9, CDKN1A, E2F1, SOS1, MAPK1, CREBS, TCF7L1, CCND1, BCL2, PDGFD, PDGFRA, GRB2, LEF1, TCF4). While many of these target genes might act as passengers, some of them are known drivers of PCa tumorigenesis. For example, somatic mutations of TP53 and RBI in PCa are well established genetic alterations [Sellers W R & Sawyers C A (2002) Somatic genetics of prostate cancer: Oncogenes and tumor suppressors, in Kantoff P W (1st ed): Prostate Cancer: Principles and Practice (Lippincott Williams & Wilkins Philadelphia, Pa., USA)]. Loss of the tumor suppressor PTEN causes activation of the PI3K-Akt signaling pathway, which is a critical oncogenic pathway in PCa [Majumder P K & Sellers W R (2005) Oncogene 24(50):7465-7474]. The PI3K-Akt path-way is an important driver of epithelial-mesenchymal transition (EMT) to reduce intercellular adhesion of cancer cells while in-creasing motility [Larue L & Bellacosa A (2005) Oncogene 24(50):7443-7454]. Recent reports suggest a crosstalk between PI3K-AKT and the androgen receptor (AR) pathway in PCa with an inactivated PTEN gene [Marques R B, et al. (2015) Eur. Urol. 67(6): 1177-1185], where activated PI3K/AKT causes PCa to become metastatic and hormone-independent. As a result, the validated miRNAs might play an important role in the regulation of aggressive PCa.

In conclusion, circulating miRNAs have been identified that serve as a molecular signature to detect VHR PCa. These biomarkers (miR-200c, miR-605, miR-135a*, miR-433, and miR-106a (sequences of each are shown in Table 11)) showed significant correlation to VHR PCa in clinical samples.

TABLE 11
Sequences of selected miRNAs disclosed
herein that are related to very high
risk (VHR) prostate cancer.

| | | SEQ |
|---|---|---|
| miRNA | Sequence (5′→3′) | ID NO |
| miR-433 | 5′-uacggugagccugucauuauuc-3′ | 1 |
| miR-200c | 5′-cgucuuacccagcaguguuugg-3′ | 2 |
| miR-106a | 5′-aaaagugcuuacagugcagguag-3′ | 4 |
| miR-135a* | 5′-uauagggauuggagccguggcg-3′ | 5 |
| miR-605 | 5′-agaaggcacuaugagauuuaga-3′ | 6 |

### Methods

Clinical Samples:

The trainings set of serum samples were purchased from two vendors as specified in Table 2 (ProteoGenex, Inc., Culver City, Calif.; and ProMedDx, LLC, Norton, Mass.). The validation set of serum samples with different grades of PCas and negative for metastasis were obtained from the NU Prostate SPORE serum repository, Chicago, Ill., following the institutional review protocol, whereas healthy serum samples were purchased from BioreclamationIVT, Baltimore, Md. Serum samples were collected from donors with matched ethnicity and sex (Caucasian and male). Samples were stored at −80° C. upon arrival and thawed on ice before use.

Isolation of Serum Exosomal RNA:

Exosomes were isolated from the discovery set of serum samples using ExoQuick™ Exosome Precipitation Solution (System Biosciences, part #EXOQ5A-1) following the manufacturer's protocol. In short, serum samples were centrifuged to remove cell debris (3000 rpm, 15 minutes). One mL serum supernatant was added to 252 μL ExoQuick™ exosome precipitation solution, mixed, and incubated at 4° C. for 30 minutes. Following incubation, the mixture was re-centrifuged and the exosome pellet was collected. RNA isolation from the exosome pellet was performed using mirVana miRNA isolation kit (Ambion, part # AM1560) following the manufacturer's protocol by suspending the exosome pellet in 300 μL of cell disruption buffer solution followed by adding 300 μL of 2× denaturing solution and was allowed to incubate on ice for 5 minutes. Next, 600 μL of acid-phenol:chloroform was added to the mixture, vortexed, and centrifuged to collect 300 μL of the aqueous phase (10,000 rpm, 5 min). The aqueous phase was mixed with 100% ethanol at a 1:1.25 volume ratio and then column filtered, followed by RNA elution with 100 μL of elution buffer. Total RNA from the filtrate was precipitated by adding 0.3 M NaCl, 20 pg glycogen, and 1 volume of isopropanol and allowed to incubate at −80° C. for 12 hrs. The mixture was centrifuged to collect the pellet (16000 rpm, 30 min, 4° C.), followed by one wash with 1 mL of 70% ethanol. The pellet was washed once with 1 mL 70% ethanol, air-dried, and suspended in 10 μL RNase-free water. Total RNA was stored at −80° C. until profiling studies using the Scano-miR bioassay.

Synthesis of the Universal SNA Nanoconjugates:

Spherical nucleic acids (SNAs) were synthesized by chemisorbing 4 μM of a propylthiol-modified ssDNA recognition sequence (5′-propylthiol-(A)₁₀-TCCTTGGTGCCCGAGTG-3′; SEQ ID NO: 3) complementary to miRNA Cloning Linker II (IDT) onto 10 nM of citrate-stabilized gold nanoparticles (13 nm in diameter) following a published protocol.27 The mixture was allowed to incubate for 1 hour at room temperature, followed by a salt ageing process consisting of 0.01% sodium dodecyl sulfate (SDS), 10 mM phosphate buffer (pH=7.4), and 0.1 M sodium chloride (NaCl), for an additional 1 hour at room temperature. Two additional aliquots of 0.1 M NaCl were added, and the mixture was allowed to incubate for 1 hour between each addition and subsequently incubated overnight (room temperature, shaking at 130 rpm). SNAs were purified through three successive rounds of centrifugation (16000×g for 20 min), supernatant removal, and re-suspension in phosphate buffered saline (137 mM NaCl, 10 mM phosphate, 2.7 mM KCl, pH 7.4). All experiments were carried out with RNase-free materials.

miRNA Profiling Using the Scano-miR Platform:

Isolated serum miRNAs were added to a ligation mixture (200 U Truncated T4 RNA Ligase 2, 900 ng miRNA cloning linker II, 12% PEG 8000, and 1× T4 RNL2 buffer) from New England Biolabs following the manufacturer's protocol, and allowed to incubate for 3 hours at 37° C. The ligation mixture was suspended in 400 μL RNase-free 2×SSC hybridization buffer (0.3 M NaCl, 0.03 M sodium citrate, pH 7.0), and hybridized onto NCode Human miRNA microarray V3 (Invitrogen) for 12 hours at 52° C. Following the incubation, the miR-array were washed to remove unbound miRNAs using pre-warmed 2×SSC (52° C.), 2×SSC, PBS (137 mM NaCl, 10 mM phosphate, 2.7 mM KCl, pH 7.4), and nanopure water. 1 nM of the synthesized SNAs suspended in 400 μL 2×SSC were hybridized onto the miR-array at 56° C. for 1 hr. The washing steps were repeated to remove unreacted SNAs. All experiments were performed using RNase-free materials. Finally, the light scattering of the gold nanoparticles was increased using three rounds of gold enhancing solution (a freshly mixed 1:1 (v:v) solution of 1 mM HAuCl₄ and 10 mM NH₂OH) (5 minutes each, at room temperature). The miR-array was imaged with a LS Reloaded scanner (Tecan, Salzburg, Austria).

Data Analysis and miRNA Clustering:

Raw Scano-miR expression data was extracted from 4,608 probes using GenePix Pro 6 software (Molecular Devices). Expression values below background threshold as well as abnormal probe shape index were filtered from data analysis. An average of three probe replicates per miRNA target was used for expression analysis. In total, 705 human miRNAs were screened for each sample. The identities and frequencies of the expression profiles were calculated for 5 exclusively expressed miRNAs that were detected solely in aggressive serum samples, where frequency denotes the number of times the miRNA was detected in the serum sample divided by the number of aggressive samples. 583 miRNAs that were not co-expressed in all 16 samples were filtered from further expression analysis. Quantile normalization was performed on 16 samples with 167 co-expressed features. Heatmaps were clustered using Pearson correlation as a distance metric and visualized using MATLAB.

Molecular Signature and Clinical Analysis:

A permutation T-test was utilized to obtain 6 differentially expressed miRNAs between Aggressive and Control samples. Permutation T-tests estimate the true null distribution of the T-test statistic. A p-value corrected for False Discovery Rate was obtained using published procedures. A molecular signature score was calculated using a published formula. The Kaplan-Meier and Wilcoxon rank sum tests were used to assess the correlation of the signature score and individual miRNA to high risk patient profiles. PCa miRNA expression data for 106 patients were downloaded from a published pilot study. The median value for each miRNA expression set was calculated. Normalized miRNA expression was dichotomized into “High” or “Low” expression of each miRNA, in relation to the median. Kaplan-Survival curves based on days to biochemical recurrence were created for each signature miRNA. Survival curve data was censored based on if the biochemical recurrence event occurred. The Mantel-Haenszel test was used to test the difference between two survival curves.

qRT-PCR Validation of the Blinded Samples.

The serum exosomes were isolated and lysed using the previously described protocol. 100 μM of synthetic cel-miR-40-3p (Applied Biosystems, part #MC10631) was spiked-in denatured exosomes. Using TaqMan RT kit (part #4366597), TaqMan hsa-miR-200c, hsa-miR-106a, hsa-miR-605, hsa-miR-371-3p, hsa-miR-135a*, hsa-miR-433, hsa-miR-495 and cel-miR-40 RT primers, 1 ng (5 μL) of total RNA from each sample were reverse transcribed in 15 μL reaction volumes following manufacturer's protocol (Applied Biosystems, TaqMan MicroRNA Assays PN 4364031 E). qRT-PCR reactions were conducted in 96 well plates with 1.33 μL of RT product with TaqMan PCR master mix (part #4364343), TaqMan probes for each miRNA in a total volume of 20 μL. An ABI Prism Model 7900 HT instrument was used to perform the qRT-PCR reactions with data analyzed using the comparative Ct method with cel-miR-40-3p utilized as an exogenous control. Known concentrations of cel-miR-40-3p were used to generate qRT-PCR standard curve. For statistical evaluation of the qRT-PCR validation test, the Mann-Whitney t-test was used, where a p-value less than 0.05 was considered statistically significant (Graph Pad Prism 6).

Sensitivity and Specificity Calculation:

The trade-off between sensitivity (true positive rate) and specificity (1-false positive rate) using the Gleason scoring sum of the first prostatic needle biopsy (FB), individual microRNA biomarkers, and molecular signature score for predicting VHR PCa, was assessed using the area under the receiver-operating characteristic (ROC) curve.

Target Genes and Pathway Analysis of the Validated miRNAs:

In silico analysis was performed in order to identify miRNA target genes and molecular pathways potentially altered by the expression of single or multiple miRNAs. Putative target genes of miRNA were determined using the homology search algorithm microT-CDS, and a database of published, experimentally-validated miRNA-gene interactions, TarBase [Reczko M, Maragkakis M, Alexiou P, Grosse I, & Hatzigeorgiou A G (2012) Bioinformatics 28(6):771-776; Vlachos I S, et al. (2015) Nucleic Acids Res. 43(Database issue):D153-159]. For microT-CDS, a microT prediction threshold of >0.8 was set. DIANA-miRPath was used to perform functional annotation clustering and pathway enrichment analysis of multiple miRNA target genes [Vlachos I S, et al. (2012) Nucleic Acids Res 40(Web Server issue):W498-504]. Two-sided Fisher's exact test and the X² test were used to classify the Gene Ontology (GO) category and KEGG pathway enrichment, and the false discovery rate (FDR) was calculated to correct p-values. A corrected p-value threshold of <0.05 was used to select significant GO categories and KEGG pathways.

The disclosure is useful in any situation where the early and rapid detection of prostate cancer with high accuracy and sensitivity is desired. Advantages provided by the disclosure include, but are not limited to:
The technology developed herein can be applied to any number of diseases where specific biomarkers are unknown—especially in the case of microRNA ones, as they are difficult to detect, especially when that are present in vanishingly small quantity.
Identified serum microRNAs can serve as diagnostic biomarkers as well as illuminate new therapeutic targets and unique pathways for prostate cancer prevention and treatment.
The use of this technology at points-of-care to diagnose, direct treatment, and monitor treatment responses for patient with prostate cancer in order to reduce costs, expedite information transfer, and to more directly diagnose, treat, and follow patients.
Identified biomarkers capable of differentiating indolent from aggressive prostate cancers in the screening setting would revolutionize patient treatment.
This uniquely positioned nanotechnology bears on an important technical problem—identifying microRNAs as biomarkers of disease for diagnosis and prevention.
There are numerous challenges associated with profiling circulating miRNAs such as the short length of miRNAs (19-25 nucleotides), the existence of sequence similarity between miRNA family members, degradative enzymes, and the presence of these biomarkers at extremely low concentrations in serum samples.
The Scano-miR system is capable of quantitatively profiling circulating miRNAs with high specificity and high sensitivity in a high-throughput fashion.
This assay, which does not rely on target enzymatic amplification and is therefore amenable to massive multiplexing, can detect such non-invasive biomarkers down to a femtomolar concentration with the capability to distinguish perfect miRNA sequences from those with single nucleotide mismatches.
The Scano-miR platform relies on the unique properties of spherical nucleic acids (SNAs) such as their high binding constant to target biomolecules and the amplifiable light scattering properties of gold nanoparticles to achieve high assay sensitivity.
The SNAs exhibit elevated melting temperatures with sharp melting transitions relative to oligonucleotide duplexes formed from traditional DNA probes of the same sequence, which can be translated into significantly higher assay specificity.
These attributes overcome many of the limitations of enzymatic amplification processes such as PCR, including without limitation the inability to screen a sample for 1000s of miRNA targets without the need to individually amplify each of the targets.
Non-invasive profiling of these miRNA biomarkers enables rapid diagnosis and accurate prediction of PCa without unnecessary surgical or treatment regimens.
The discovery and identification of novel biomarkers for PCa diagnosis is necessary in order to address the inaccuracies of PSA screening and Gleason scoring based solely on prostatic needle biopsy specimens.
The disclosure demonstrates that the miRNAs identified by the Scano-miR bioassay are at least 94% accurate in differentiating between aggressive versus indolent PCa, while the prostatic needle biopsy Gleason grading technique is only 77% accurate.

The discovery and identification of the panel of miRNAs for PCa diagnosis disclosed herein will revolutionize how urologists screen patients for prostate cancer and how to accurately interpret the results in order to address the inaccuracies of the current PSA screening and Gleason scoring based solely on prostatic needle biopsy specimens.

### REFERENCE

(1) Jemal, A.; Siegel, R.; Ward, E.; Murray, T.; Xu, J.; Thun, M. J. CA. Cancer J. Clin. 2007, 57, 43.
(2) Heijnsdijk, E. A.; der Kinderen, A.; Wever, E. M.; Draisma, G.; Roobol, M. J.; de Koning, H. J. Br. J. Cancer 2009, 101, 1833.
(3) Grubb, R. L., 3rd; Black, A.; Izmirlian, G.; Hickey, T. P.; Pinsky, P. F.; Mabie, J. E.; Riley, T. L.; Ragard, L. R.; Prorok, P. C.; Berg, C. D.; Crawford, E. D.; Church, T. R.; Andriole, G. L., Jr. Cancer Epidemiol. Biomarkers Prev. 2009, 18, 748.
(4) Andriole, G. L.; Crawford, E. D.; Grubb, R. L., 3rd; Buys, S. S.; Chia, D.; Church, T. R.; Fouad, M. N.; Gelmann, E. P.; Kvale, P. A.; Reding, D. J.; Weissfeld, J. L.; Yokochi, L. A.; O'Brien, B.; Clapp, J. D.; Rathmell, J. M.; Riley, T. L.; Hayes, R. B.; Kramer, B. S.; Izmirlian, G.; Miller, A. B.; Pinsky, P. F.; Prorok, P. C.; Gohagan, J. K.; Berg, C. D. N. Engl. J. Med. 2009, 360, 1310.
(5) Crawford, E. D.; Andriole, G. L.; Marberger, M.; Rittmaster, R. S. Urology 2010, 75, 502.
(6) Schroder, F. H.; Hugosson, J.; Roobol, M. J.; Tammela, T. L.; Ciatto, S.; Nelen, V.; Kwiatkowski, M.; Lujan, M.; Lilja, H.; Zappa, M.; Denis, L. J.; Recker, F.; Berenguer, A.; Maattanen, L.; Bangma, C. H.; Aus, G.; Villers, A.; Rebillard, X.; van der Kwast, T.; Blijenberg, B. G.; Moss, S. M.; de Koning, H. J.; Auvinen, A. N. Engl. J. Med. 2009, 360, 1320.
(7) Epstein, J. I. J. Urol. 2010, 183, 433.
(8) Hugosson, J.; Carlsson, S.; Aus, G.; Bergdahl, S.; Khatami, A.; Lodding, P.; Pihl, C. G.; Stranne, J.; Holmberg, E.; Lilja, H. The lancet oncology 2010, 11, 725.
(9) Schroder, F. H.; Hugosson, J.; Roobol, M. J.; Tammela, T. L.; Ciatto, S.; Nelen, V.; Kwiatkowski, M.; Lujan, M.; Lilja, H.; Zappa, M.; Denis, L. J.; Recker, F.; Paez, A.; Maattanen, L.; Bangma, C. H.; Aus, G.; Carlsson, S.; Villers, A.; Rebillard, X.; van der Kwast, T.; Kujala, P. M.; Blijenberg, B. G.; Stenman, U. H.; Huber, A.; Taari, K.; Hakama, M.; Moss, S. M.; de Koning, H. J.; Auvinen, A. N. Engl. J. Med. 2012, 366, 981.
(10) Conti, S. L.; Dall'era, M.; Fradet, V.; Cowan, J. E.; Simko, J.; Carroll, P. R. J. Urol. 2009, 181, 1628.
(11) Dall'Era, M. A.; Carroll, P. R. Curr. Opin. Urol. 2009, 19, 258.
(12) Bostwick, D. G.; Myers, R. P.; Oesterling, J. E. Semin. Surg. Oncol. 1994, 10, 60.
(13) Isariyawongse, B. K.; Sun, L.; Banez, L. L.; Robertson, C.; Polascik, T. J.; Maloney, K.; Donatucci, C.; Albala, D.; Mouraviev, V.; Madden, J. F.; Moul, J. W. Urology 2008, 72, 882.
(14) Chun, F. K.; Epstein, J. I.; Ficarra, V.; Freedland, S. J.; Montironi, R.; Montorsi, F.; Shariat, S. F.; Schroder, F. H.; Scattoni, V. Eur. Urol. 2010, 58, 851.
(15) Lee, R. C.; Feinbaum, R. L.; Ambros, V. Cell 1993, 75, 843.
(16) Lim, L. P.; Lau, N. C.; Garrett-Engele, P.; Grimson, A.; Schelter, J. M.; Castle, J.; Bartel, D. P.; Linsley, P. S.; Johnson, J. M. Nature 2005, 433, 769.
(17) Lewis, B. P.; Burge, C. B.; Bartel, D. P. Cell 2005, 120, 15.
(18) Mitchell, P. S.; Parkin, R. K.; Kroh, E. M.; Fritz, B. R.; Wyman, S. K.; Pogosova-Agadjanyan, E. L.; Peterson, A.; Noteboom, J.; O'Briant, K. C.; Allen, A.; Lin, D. W.; Urban, N.; Drescher, C. W.; Knudsen, B. S.; Stirewalt, D. L.; Gentleman, R.; Vessella, R. L.; Nelson, P. S.; Martin, D. B.; Tewari, M. Proc. Natl. Acad. Sci. USA 2008, 105, 10513.
(19) Selth, L. A.; Townley, S.; Gillis, J. L.; Ochnik, A. M.; Murti, K.; Macfarlane, R. J.; Chi, K. N.; Marshall, V. R.; Tilley, W. D.; Butler, L. M. Int. J. Cancer 2012, 131, 652.
(20) Valadi, H.; Ekstrom, K.; Bossios, A.; Sjostrand, M.; Lee, J. J.; Lotvall, J. O. Nature Cell Biol. 2007, 9, 654.
(21) Yaman Agaoglu, F.; Kovancilar, M.; Dizdar, Y.; Darendeliler, E.; Holdenrieder, S.; Dalay, N.; Gezer, U. Tumour Biol. 2011, 32, 583.
(22) Bryant, R. J.; Pawlowski, T.; Catto, J. W.; Marsden, G.; Vessella, R. L.; Rhees, B.; Kuslich, C.; Visakorpi, T.; Hamdy, F. C. Br. J. Cancer 2012, 106, 768.
(23) Ohshima, K.; Inoue, K.; Fujiwara, A.; Hatakeyama, K.; Kanto, K.; Watanabe, Y.; Muramatsu, K.; Fukuda, Y.; Ogura, S.; Yamaguchi, K.; Mochizuki, T. PloS one 2010, 5, e13247.
(24) Gallo, A.; Tandon, M.; Alevizos, I.; Illei, G. G. PloS one 2012, 7, e30679.
(25) Tanaka, Y.; Kamohara, H.; Kinoshita, K.; Kurashige, J.; Ishimoto, T.; Iwatsuki, M.; Watanabe, M.; Baba, H. Cancer 2013, 119, 1159.
(26) Grasedieck, S.; Sorrentino, A.; Langer, C.; Buske, C.; Dohner, H.; Mertens, D.; Kuchenbauer, F. Blood 2013, 121, 4977.
(27) Alhasan, A. H.; Kim, D. Y.; Daniel, W. L.; Watson, E.; Meeks, J. J.; Thaxton, C. S.; Mirkin, C. A. Anal. Chem. 2012, 84, 4153.
(28) Mirkin, C. A.; Letsinger, R. L.; Mucic, R. C.; Storhoff, J. J. Nature 1996, 382, 607.
(29) Taton, T. A.; Mirkin, C. A.; Letsinger, R. L. Science 2000, 289, 1757.
(30) Cutler, J. I.; Auyeung, E.; Mirkin, C. A. J. Am. Chem. Soc. 2012, 134, 1376.
(31) Cao, Y. C.; Jin, R.; Mirkin, C. A. Science 2002, 297, 1536.
(32) Zeng, X.; Xiang, J.; Wu, M.; Xiong, W.; Tang, H.; Deng, M.; Li, X.; Liao, Q.; Su, B.; Luo, Z.; Zhou, Y.; Zhou, M.; Zeng, Z.; Li, X.; Shen, S.; Shuai, C.; Li, G.; Fang, J.; Peng, S. PloS one 2012, 7, e46367.
(33) Dijkstra, S.; Hamid, A. R.; Leyten, G. H.; Schalken, J. A. Prostate cancer 2012,2012, 327104.
(34) Kaplan, E. L.; Meier, P. J. Amer. Statist. Assn. 1958, 53, 457.
(35) Mann, H. B.; Whitney, D. R. Annals of Math. Stat. 1947, 18, 50.
(36) Watahiki, A.; Macfarlane, R. J.; Gleave, M. E.; Crea, F.; Wang, Y.; Helgason, C. D.; Chi, K. N. Int J Mol Sci 2013, 14, 7757.
(37) Volinia, S.; Calin, G. A.; Liu, C. G.; Ambs, S.; Cimmino, A.; Petrocca, F.; Visone, R.; Iorio, M.; Roldo, C.; Ferracin, M.; Prueitt, R. L.; Yanaihara, N.; Lanza, G.; Scarpa, A.; Vecchione, A.; Negrini, M.; Harris, C. C.; Croce, C. M. Proc. Natl. Acad. Sci. U.S.A. 2006, 103, 2257.
(38) Holleman, A.; Chung, I.; Olsen, R. R.; Kwak, B.; Mizokami, A.; Saijo, N.; Parissenti, A.; Duan, Z.; Voest, E. E.; Zetter, B. R. Oncogene 2011, 30, 4386.
(39) Huang, S. P.; Levesque, E.; Guillemette, C.; Yu, C. C.; Huang, C. Y.; Lin, V. C.; Chung, I. C.; Chen, L. C.; Laverdiere, I.; Lacombe, L.; Fradet, Y.; Chang, T. Y.; Lee, H. Z.; Juang, S. H.; Bao, B. Y. Int. J. Cancer 2014, 135, 2661.

What is claimed is:

1. A method of detecting aggressive prostate cancer in an individual, the method comprising: isolating miRNA from a sample from the individual; ligating the miRNA to a universal linker; hybridizing the miRNA to a nucleic acid that is on a surface, wherein the nucleic acid is complementary to miR-433 and/or miR-200c; contacting the miRNA with a spherical nucleic acid (SNA), wherein the SNA comprises a polynucleotide that is sufficiently complementary to the universal linker to hybridize under appropriate conditions; wherein detection of the SNA is indicative of aggressive prostate cancer in the individual.

2. The method of claim 1 wherein the SNA comprises a metal.

3. The method of claim 2 wherein the SNA comprises gold.

4. The method of claim 1 wherein the SNA is hollow.

5. The method of claim 1 wherein the SNA comprises a liposome.

6. The method of any one of claims 1-5 wherein the surface is an array.

7. The method of claim 6 wherein the array comprises a plurality of different nucleic acids.

8. The method of any one of claims 1-7 wherein the sample is a body fluid, serum, or tissue obtained from an individual suffering from a disease.

9. The method of any one of claims 1-7 wherein the sample is a liquid biopsy obtained from an individual suffering from a disease.

10. The method of any one of claims 1-7 wherein the sample is a body fluid, serum, or tissue obtained from an individual not known to be suffering from a disease.

11. The method of any one of claims 1-7 wherein the sample is a liquid biopsy obtained from an individual not known to be suffering from a disease.

12. The method of claim 8 or claim 9 wherein the profile is compared to an earlier profile determined from the individual.

13. The method of claim 10 or claim 11 wherein the profile is compared to a profile determined from an additional individual known to be suffering from aggressive prostate cancer.

14. The method of any one of claims 1-13 wherein the miRNA is exosomal miRNA.

15. The method of any one of claims 1-14 wherein the aggressive prostate cancer is very high risk prostate cancer.
